(19) United States
(12) Patent Application Publication
(10) Pub. No.: US 2012/0283530 A1
(43) Pub. Date: Nov. 8, 2012

(54) METHOD AND APPARATUS TO DETECT CORONARY ARTERY CALCIFICATION OR DISEASE

(76) Inventors: John D. Maynard, Albuquerque, NM (US); Nathaniel Matter, Albuquerque, NM (US); Baqiyyah Conway, Hermitage, TN (US); Trevor Orchard, Monroeville, PA (US)

(21) Appl. No.: 13/509,871

(22) PCT Filed: Nov. 17, 2010
(86) PCT No.: PCT/US2010/057092

§ 371 Date: Jul. 3, 2012

Related U.S. Application Data
(60) Provisional application No. 61/261,919, filed on Nov. 17, 2009.

(51) Int. Cl. A61B 5/1455; A61B 6/00

(52) U.S. Cl. 600/316; 600/479; 600/322

(57) ABSTRACT

Coronary artery calcification (CAC) occurs at an earlier age in diabetes and is a risk factor for coronary artery disease (CAD) in subjects with or without diabetes. One postulated mechanism for the increased CAC is the accelerated accumulation of advanced glycation end products (AGEs) in the vasculature. As certain collagen AGEs fluoresce, skin intrinsic fluorescence (SIF) can act as a novel maker of collagen AGEs levels. The present invention provides methods and apparatuses for detecting SIF that can be a useful marker of CAD risk and a therapeutic target.

### FIELD OF THE INVENTION

The present invention generally relates to determination of a tissue state from the response of tissue to incident light. More specifically, the present invention relates to a method and apparatus to detect coronary artery calcification using skin intrinsic fluorescence of an individual.

### BACKGROUND ART

Coronary artery disease (CAD) is the leading cause of death in patients with and without diabetes; however, risk factors for CAD in these populations are not completely understood. Coronary artery calcification (CAC), more severe and occurring at an earlier age in type 1 and type 2 diabetes, is a subclinical marker of atherosclerotic burden and correlated with prevalent and future clinical coronary artery disease events. See D. Dabelea et al., “The Coronary Artery Calcification in Type 1 Diabetes (CACTI) Study,” Diabetes 52:2833-9, 2003; J. Rumberger et al., “Electron-beam tomographic coronary calcium scanning: a review and guidelines for use in asymptomatic persons,” Mayo Clin Proc 74:243-52, 1999; J. Olson et al., “Coronary calcium in adults with type 1 diabetes: a stronger correlate of clinical coronary artery disease in men than in women,” Diabetes 49:1571-8, 2000; Y Arad et al., “Prediction of coronary events with electron-beam tomography,” J Am Coll Cardiol 36:1253-60, 2000; P. Raggi et al., “Identification of patients at increased risk of unheralded myorcardial infarction by Electron-Beam Computed Tomography,” Circulation 101:850-5, 2000; J. Rumberger et al., “Coronary Calcium, as Determined by Electron Beam Computed Tomography, and Coronary Disease on Arteriogram,” Circulation 91:1363-7, 1995; and R. Detrano et al., “Coronary calcium as a predictor of coronary events in four racial or ethnic groups,” New England Journal of Medicine 358:1336-45, 2008. One postulated mechanism for the increased CAC observed in patients with or without diabetes is the accumulation of advanced glycation end products (AGEs). Both AGEs and CAC are increased in patients with diabetes, and AGEs have been shown to induce osteoblastic differentiation of microvascular pericytes, thereby increasing vascular calcification. See Z. Makita et al., “Reactive glycosylation end products in diabetic uraemia and treatment of renal failure,” Lancet 343:1519-22, 1994; A. Dawnay and D. Millar, “The pathogenesis and consequences of AGE formation in uraemia and its treatment,” Cell Mol Biol 44:1081-94, 1998; N. Verzijl et al., “Effect of Collagen Turnover on the Accumulation of Advanced Glycation End Products,” J Biol Chem 50:39027-31, 2000; and S. Yamagishi et al., “Advanced glycation end products accelerate calcification in microvascular pericytes,” Biochemical and Biophysical Communications 258:353-7, 1999.

AGEs are macroprotein complexes formed by the Malliard reaction of reducing sugars with free amino groups on proteins, amino acids, or lipids. Many AGEs form molecular cross-links and fluoresce. As certain dermal collagen AGEs, such as pentosidine and crosslines, contain fluorescent crosslinks, skin intrinsic fluorescence can be quantified and act as a novel maker of collagen AGEs. See V. Monnier et al., “Skin Collagen Glycation, Glycoxidation, and Crosslinking Are Lower in Subjects with Long-term Intensive verses Conventional Therapy of Type 1 Diabetes,” Diabetes 48:870-80, 1999. Skin intrinsic fluorescence, determined by the SCOUT DM skin fluorescence reader from VeraLight, Inc. (SCOUT, SCOUT DM, and VERALIGHT are trademarks of VeraLight, Inc.), was recently found to be cross-sectionally associated with neuropathy, micro- and macroalbuminuria, CAC, and marginally with CAD in a preliminary analysis of 47 participants of the Pittsburgh Epidemiology of Diabetes Complications (EDC) study. See B. W J Conway et al., “Skin Fluorescence and Type 1 Diabetes: A New Marker of Complication Risk,” Diabetes 57(S1):A287, 2008. However, only for CAC and neuropathy was a relationship observed independent of renal function, which is important since accumulation of AGEs and CAC is greatly accelerated in renal disease and renal function is tightly tied to AGE clearance. See K. Taki et al., “Oxidative stress, advanced glycation end product, and coronary artery calcification in hemodialysis patients,” Kidney Int 70:218-24, 2006; and Z. Makita et al., “Advanced glycation end products in patients with diabetic nephropathy,” New England Journal of Medicine 325:836-42, 1991.

In addition, a study of 155 participants at the New Mexico Heart Institute showed a significant relationship between skin fluorescence and degree of coronary artery calcification. Of the 155 participants, 143 did not have diabetes and this study is one of the first to show a relationship between skin fluorescence and CAC in subjects without diabetes.

A noninvasive method and apparatus for detecting disease in an individual using fluorescence spectroscopy and multivariate analysis has been previously disclosed in U.S. Pat. No. 7,139,598, incorporated herein by reference. Continued development of this method and apparatus has resulted in significant instrument and algorithm improvements that yield increased accuracy for noninvasively detecting disease, especially type 2 diabetes and pre-diabetes. The instrument improvements provide higher overall signal to noise ratio, reduced measurement time, better reliability, tighter precision, lower cost and reduced size compared to instruments disclosed in the art. The algorithmic improvements increase overall accuracy by more effective extraction of the information needed for accurate noninvasive detection of disease using fluorescence spectroscopy.

Accordingly, the present invention provides methods and apparatuses to noninvasively measure skin intrinsic fluorescence and CAC in an individual to enable objective determination of coronary artery disease risk.

### DISCLOSURE OF INVENTION

The present invention provides methods and apparatuses to noninvasively detect coronary artery calcification in an individual. The method comprises providing a spectroscopic apparatus adapted to measure the skin fluorescence of the individual and detecting the skin fluorescence of the individual with the spectroscopic apparatus.

Skin intrinsic fluorescence can be measured with a spectroscopic apparatus suitable for determining properties of in vivo tissue from spectral information collected from the tissue. An illumination system provides light at a plurality of broadband ranges, which are communicated to an optical probe. The optical probe receives light from the illumination system and transmits it to in vivo tissue, and receives light diffusely reflected in response to the broadband light, emitted from the in vivo tissue by fluorescence thereof in response to the broadband light, or a combination thereof. The optical probe communicates the light to a spectrograph which produces a signal representative of the spectral properties of the light. An analysis system determines a property of the in vivo tissue from the spectral properties. A calibration device mounts such that it is periodically in optical communication with the optical probe.

The apparatus can be used for determining a disease state, such as the presence coronary artery calcification, coronary artery disease, or a combination thereof, from spectral information collected from the tissue. An illumination system provides light at a plurality of broadband ranges, which are communicated to an optical probe. The optical probe receives light from the illumination system and transmits it to in vivo tissue, and receives light diffusely reflected in response to the broadband light, emitted from the in vivo tissue by fluorescence thereof in response to the broadband light, or a combination thereof. The optical probe communicates the light to a spectrograph which produces a signal representative of the spectral properties of the light. An analysis system determines a property of the in vivo tissue from the spectral properties. A calibration device mounts such that it is periodically in optical communication with the optical probe.

The apparatus can include a plurality of light emitting diodes (LEDs) or laser diodes in the illumination system, and can include at least one filter that substantially rejects light from the LEDs that has the same wavelength of a wavelength of light fluoresced by materials of interest in the tissue. Some embodiments include one or more light pipes that encourage uniform illumination by the illumination system or by the optical probe. Some embodiments include movably mounted LEDs or laser diodes, such as by rotation of a carrier, to allow selective coupling of different LEDs or laser diodes to the optical probe. Some embodiments include real-time monitoring of the light generated by the illumination system to allow compensation for time and/or temperature-dependent changes in the amount of light generated. Some embodiments include specific operator displays, including operator displays that incorporate a touchscreen interface. Some embodiments include optical fibers in the optical probe, which fibers are arranged to provide specific relationships between illumination of the tissue and collection of light from the tissue. Some embodiments include a spectrograph which produces a signal representative of the spectral properties of light that is free from artifacts such as ghost images and excess stray light. Some embodiments incorporate a calibration device that may contain fluorescent material and allows measurement of reflectance and/or emitted fluorescence.

### BRIEF DESCRIPTION OF DRAWINGS

The accompanying drawings, which are incorporated in and form part of the specification, illustrate the present invention and, together with the description, describe the invention. In the drawings, like elements are referred to by like numbers.

FIG. 1 is an illustration of an example spectroscopic apparatus that can be used to measure skin intrinsic fluorescence.

FIG. 2 is an illustration of an example spectroscopic apparatus that can be used to measure skin intrinsic fluorescence.

FIG. 3 is a schematic depiction of an illumination system suitable for use in the present invention.

FIG. 4 is a schematic isometric view of an illumination system suitable for use in the present invention.

FIG. 5 is a schematic isometric view of an illumination system suitable for use in the present invention.

FIG. 6 is an illustration of an array of light emitting diodes suitable for use in an illumination system in the present invention.

FIG. 7 is a schematic depiction of an optical probe suitable for use in the present invention.

FIG. 8 is a schematic depiction of an optical probe suitable for use in the present invention, seen from the interface with the tissue.

FIG. 9 is an illustration of a cradle and calibration device of an embodiment of the present invention.

FIG. 10 is a flow diagram of a method of determining disease classification according to the present invention.

FIG. 11a is a front isometric view of an illumination system suitable for use in the present invention.

FIG. 11b is a back isometric view of an illumination system suitable for use in the present invention.

FIG. 12 is an isometric view of a portion of a wheel assembly suitable for use in the example illumination system of FIG. 11a and FIG. 11b.

FIG. 13 is a schematic cross-sectional view of an illumination system having the two illumination channels.

FIG. 14 is an isometric view of an example embodiment of a trifurcated optical probe having two input illumination channels and one detection channel.

FIG. 15 is a schematic depiction of optical fibers in an example optical probe according to the present invention, providing two different illumination-collection characteristics.

FIG. 16 is a schematic depiction of an example spectrograph suitable for use in the present invention.

FIG. 17 is an illustration of an example image formed onto a CCD image sensor with multiple wavelengths of 360, 435, 510, 585, and 660 nm, and the corresponding spectrum produced by vertically binning the pixels of the CCD.

FIG. 18 is a schematic depiction of an example spectrograph suitable for use in the present invention.

FIG. 19 is a schematic depiction of an example spectrograph suitable for use in the present invention.

FIG. 20 is an illustration of an example embodiment of an apparatus according to the present invention.

FIG. 21 is an illustration of a comparison of OGTT and FPG screening categorization obtained using the present invention.

FIG. 22 is an illustration of receiver-operator characteristics obtained using the present invention.

FIG. 23 illustrates aggregate results of the effect of data regularization according to the present invention on the skin fluorescence spectra in terms of sensitivity to disease with respect to SVR classification.

FIG. 24 illustrates results of the effect of data regularization for an individual sub-model for male/dark skin.

FIG. 25 illustrates results of the effect of data regularization for an individual sub-model for male/light skin.

FIG. 26 illustrates results of the effect of data regularization for an individual sub-model for female/dark skin.

FIG. 27 illustrates results of the effect of data regularization for an individual sub-model for female/light skin.

FIG. 28 is an illustration of the age dependence of skin fluorescence.

FIG. 29 is an illustration of skin color monitoring.

FIG. 30 is an illustration of a receiver operator characteristic relating to optical separation of genders.

FIG. 31 is an illustration of a receiver operator characteristic relating to detection of impaired glucose tolerance.

FIG. 32 is an illustration of a receiver operator characteristic relating to detection of impaired glucose tolerance.

FIG. 33 is a schematic diagram of an example LED driver circuit suitable for use with some embodiments of the present invention.

FIG. 34 is a schematic illustration of an example light source subsystem useful in some embodiments of the present invention.

FIG. 35 is a schematic diagram of a circuit useful in connection with some example embodiments of the present invention.

FIG. 36 is an illustration of examples of the output energy drift of six different LEDs due to intentional perturbation of the ambient temperature.

FIG. 38(A,B,C) are schematic illustrations of example calibration maintenance devices suitable for use with some embodiments of the present invention.

FIG. 39 is an illustration of a two-dimensional diffraction pattern created by the two-dimensional structure of a CCD pixel array.

FIG. 40 is an illustration of tissue reflectance and fluorescence spectrum with reflected excitation and a superimposed excitation “ghost”.

FIG. 41 is a schematic illustration of an out-of-plane Littrow mount design suitable for use in some embodiments of the present invention.

FIG. 42 is an end-on view looking toward the concave surface of the grating.

FIG. 43 is an illustration of the absorption coefficients of melanin, hemoglobin, water and protein (i.e. collagen, elastin) over the 150 nm to 1100 nm spectral region.

FIG. 44 is a graph showing the median (log 10) coronary artery calcification by tertiles of skin intrinsic fluorescence.

FIG. 45 is a collection of graphs of receiver operator characteristic curves for the detection of coronary artery calcification (CAC) in patients with type 1 diabetes with a total volume CAC score >0, >200, and >400.

FIG. 46 is a graph showing the receiver operator characteristic curves for the detection of coronary artery calcification with a simple skin intrinsic fluorescence sum from a patient cohort largely without diabetes with a total volume CAC score >200.

FIG. 47 is a graph showing the receiver operator characteristic curve for detection of CAC using multivariate linear discriminate analysis of skin intrinsic fluorescence spectra from a patient cohort largely without diabetes with a total volume CAC score >20.

### MODES FOR CARRYING OUT THE INVENTION AND INDUSTRIAL APPLICABILITY

The present invention uses an association between skin intrinsic fluorescence, a marker of skin collagen AGEs, and CAC. Increased levels of AGEs have been associated with arterial calcification of the coronary arteries in hemodialysis patients, with medial wall calcification of the internal thoracic artery of diabetic patients with coronary artery disease, and with medial wall calcification of the limbs of diabetic patients with neuropathy. See K. Taki et al., “Oxidative stress, advanced glycation end product, and coronary artery calcification in hemodialysis patients,” Kidney Int 70:218-24, 2006; N. Sakata et al., “Calcification of the Medial Layer of the Internal Thoracic Artery in Diabetic Patients: Relevance of Glycoxidation,” J Vasc Res 40:467-574, 2003; M. Edmonds et al., “Medial arterial calcification and diabetic neuropathy,” British Medical Journal 284:928-30, 1982; and F. Goebel and H. Fuessl, “Mockenberg's sclerosis after sympathetic denervation in diabetic and non-diabetic subjects,” Diabetologia 24:347-50, 1983. The method of the present invention uses an age- and renal damage-independent relationship between skin intrinsic fluorescence, a marker of AGEs, and the severity of calcification of the coronary arteries. The method also uses a relationship with the progression of CAC, independent of age and renal function (serum creatinine) and renal damage (albumin excretion rate). The method further uses an association between skin intrinsic fluorescence and CAC at clinically significant thresholds associated with coronary artery disease.

Study of the Association of Skin Intrinsic Fluorescence with Coronary Artery Calcification

The Epidemiology of Diabetes Complications Study (EDC) cohort is a well defined population (n=658) of type 1 diabetes diagnosed before the age of seventeen years at Children's Hospital of Pittsburgh. See T. Orchard et al., “The prevalence of complications in insulin-dependent diabetes mellitus by sex and duration: Pittsburgh Epidemiology of Diabetes Complications Study-II,” Diabetes 39:1116-24, 1990; and T. Orchard et al., “Factors associated with the avoidance of severe complications after 25 years of insulin-dependent diabetes mellitus: Pittsburgh Epidemiology of Diabetes Complications Study-I,” Diabetes Care 13:741-7, 1990. Mean age and diabetes duration at study baseline (1986-1988) were 28 and 19 years, respectively. Participants have been followed biennially by survey and medical exam. One hundred and five participants (96% Caucasian) from the EDC study who had previously undergone electron beam tomography (EBT, GE-Imatron C-150) scanning for coronary artery calcification (CAC) in either the sixteenth or eighteenth year of follow-up consented to participate in the Noninvasive Skin Spectroscopy Substudy for Diabetes Complications, a cross-sectional study, during the 20^{th} year follow-up period. All procedures were approved by the Institutional Review Board of the University of Pittsburgh.

Eighty participants had previously had CAC measured during the ten year follow-up period (1996-1998) and were included in a sub-analysis of CAC progression. Threshold calcium determination was set using a density of 130 Hounsfield units in a minimum of 2 contiguous sections of the heart. Scans were triggered by ECG signals at 80% of the R-R interval. Total volume CAC scores were calculated based on isotropic interpolation and were used for analyses. See T. Callister et al., “Coronary artery disease: improved reproducibility of calcium scoring with electron beam CT volumetric method,” Radiology 208:807-14, 1998. Skin intrinsic fluorescence covariate data were collected, as previously described, at the time of CAC assessment. See T. Orchard et al., “The prevalence of complications in insulin-dependent diabetes mellitus by sex and duration: Pittsburgh Epidemiology of Diabetes Complications Study-II,” Diabetes 39:1116-24, 1990; and T. Orchard et al., “Factors associated with the avoidance of severe complications after 25 years of insulin-dependent diabetes mellitus: Pittsburgh Epidemiology of Diabetes Complications Study-I,” Diabetes Care 13:741-7, 1990. Blood samples were assayed for lipids, lipoproteins, glycosylated hemoglobin, and creatinine. High-density lipoprotein (HDL) cholesterol was determined by a heparin and manganese procedure, a modification of the Lipid Research Clinics method. See G. Warnick and J. Albers, “Heparin-Mn2+ quantification of high density lipoprotein cholesterol: An ultrafiltration procedure for lipemic samples,” Clin Chem 24:900-4, 1978; and National Institutes of Health DoH, Education, and Welfare, Lipid Research Clinics Program (NIH pub no. 75-628) Washington, D.C.: U.S. Government Printing Office; 1975. Cholesterol was measured enzymatically. See C. Allain et al., “Enzymatic determination of total serum cholesterol,” Clin Chem 20:470-5, 1974. Non-high density lipoprotein cholesterol was calculated as the difference between the total cholesterol and the high density lipoprotein cholesterol. Original A_{1c} was measured with the DCA 2000 analyzer (Bayer, Tarrytown, N.Y., USA) and converted to Diabetes Control and Complications Trial (DCCT) aligned HbA_{1c} values using regression formulas derived from duplicate analyses (DCCT HbA_{1c}=[EDC HbA_{1c}−1.13]/0.81). Urinary albumin was determined immunonephelometrically. Height was measured using a stadiometer and weight using a balance beam scale. Body mass index (BMI) was calculated as the weight in kilograms divided by the square of the height in meters. Blood pressure was measured by a random-zero sphygmomanometer according to a standardized protocol after a 5-minute rest period. Blood pressure levels were analyzed, using the mean of the second and third readings. A history of CAD was defined as myocardial infarction, ischemia (Minnesota Codes 1.1-1.3, 4.1-4.3, 5.1-5.3 and 7.1), revascularization, or EDC clinic diagnosed angina.

Skin intrinsic fluorescence was non-invasively measured from the skin of the volar forearm using three SCOUT DM (VeraLight, Inc., Albuquerque, N. Mex.) skin fluorescence spectrometers, as described in more detail below. Skin fluorescence was excited with a LED light source centered at 375 nm and was detected over the range of 441-496 nm. The skin reflectance was measured over both the excitation and emission regions and was used to compensate for absorbance due to melanin and hemoglobin. The intrinsic fluorescence correction is expressed in following equation,

$$f ( λ ) = \frac{F ( λ )}{{R}_{x}^{{k}_{x}} {{R}_{m} ( λ )}^{{k}_{m}}} \qquad a .$$

where λ is the emission wavelength. See E. Hull et al., “Noninvasive, optical detection of diabetes: model studies with porcine skin,” Opt Express 12:4496-510, 2004. The measured fluorescence, F(A), is divided by reflectance values at the excitation and emission wavelengths, Rₓ and Rₘ (λ), respectively. The reflectance values are adjusted by the dimensionless exponents, kₓ and kₘ. See B. W J Conway et al., “Skin Fluorescence and Type 1 Diabetes: A New Marker of Complication Risk,” Diabetes 57(S1):A287, 2008. For these analyses, kₓ=0.6 and kₘ=0.2. The resulting intrinsic fluorescence, f(λ), was integrated over the 441 to 496 nm spectral region to give the skin intrinsic fluorescence score.

Statistical Analyses

CAC volume scores were natural logarithmically transformed after adding 1 to their value. The student's t test and chi-square tests were used to examine univariate correlates of CAC prevalence. Logistic regression analysis with stepwise selection was used to determine the independent association of skin intrinsic fluorescence with the prevalence of coronary artery calcification. Receiver operator characteristic (ROC) curves were used to determine the discriminative ability of skin intrinsic fluorescence to detect CAC at thresholds of total volume CAC score of >0, >200, and >400. Spearman's correlation was used to determine the association of skin intrinsic fluorescence with the severity of CAC, i.e. the total volume CAC score. Linear regression analysis with stepwise selection was used to determine the independent association of skin intrinsic fluorescence with the severity of CAC. For the analysis of CAC progression, progression was defined as >2.5 change in the square-root transformed CAC score. See J. Hokanson et al., “Evaluating changes in coronary artery calcium: an analytic method that accounts for interscan variability,” AM J Roentgenol 182:1327-32, 2004. Odds ratios and regression coefficients are expressed as per standard deviation change in the continuous variables.

Results of Study

Characteristics of the study participants by the prevalence of CAC (at latest assessment) are presented in Table 1. Seventy-one percent of the study participants had some measureable CAC. Participants with CAC were older, of longer diabetes duration, had a higher skin intrinsic fluorescence, a higher albumin excretion rate, a greater prevalence of long term complications of diabetes, had a marginally lower diastolic blood pressure, and marginally more likely to be on lipid or hypertension medication. In Table 1, * signifies Total volume calcification score; ** signifies Fisher's exact p-value; risk factors, with the exception of skin intrinsic fluorescence, were measured at the time of CAC assessment.

TABLE 1

| | CAC positive | | CAC negative | | |
|---|---|---|---|---|---|
| Characteristic | (n = 75) | | (n = 30) | | p-value |
| Skin intrinsic fluorescence | 0.033 | (0.007) | 0.028 | (0.006) | 0.0009 |
| Age (yrs) | 46.4 | (6.6) | 39.1 | (6.5) | <0.0001 |
| Diabetes duration (yrs) | 36.7 | (6.7) | 31.5 | (7.2) | 0.0006 |
| Sex (female) | 57.33 | (43) | 53.33 | (16) | 0.71 |
| HbA1c (%) (n = 74; 28) | 7.4 | (1.2) | 7.6 | (1.4) | 0.38 |
| Body mass index (BMI (kg/m²) (74; 30) | 26.17 | (4.64) | 25.14 | (3.06) | 0.18 |
| Serum creatinine* (mg/dl) (n = 74; 29) | 1.0 | (0.8-1.2) | 0.9 | (0.8-0.9) | 0.14 |
| Albumin excretion rate* (μg/min) (n = 73; 28) | 8.4 | (4.0-47.3) | 4.1 | (2.3-7.5) | 0.004 |
| HDL cholesterol (mg/dl) (n = 73; 29) | 59.5 | (17.0) | 59.4 | (15.1) | 0.99 |
| Non-HDL cholesterol (mg/dl) (n = 73; 29) | 124.5 | (28.0) | 125.9 | (25.6) | 0.82 |
| Systolic blood pressure (mmHg) (n = 72; 29) | 118.8 | (16.9) | 116.0 | (10.3) | 0.31 |
| Diastolic blood pressure (m Hg) (n = 72; 29) | 66.3 | (9.3) | 70.1 | (9.7) | 0.07 |
| Coronary artery disease | 36.0 | (27) | 16.7 | (5) | 0.05 |
| Proliferative Retinopathy | 64.0 | (48) | 26.7 | (8) | 0.0005 |
| Overt Nephropathy | 32.0 | (24) | 10.0 | (3) | 0.03** |
| Distal Symmetrical Polyneuropathy | 69.3 | (52) | 30.0 | (9) | 0.0002 |
| Lower Extremity Arterial Disease | 40.0 | (3) | 10.0 | (3) | 0.002** |
| History of smoking | 32.9 | (24) | 24.1 | (7) | 0.39 |
| ACE/ARB medication use (n = 73; 29) | 49.3 | (36) | 27.6 | (8) | 0.05 |
| Statin use (n = 73; 29) | 42.5 | (31) | 20.7 | (6) | 0.04 |

Univariate logistic regression analysis revealed that each standard deviation change in skin intrinsic fluorescence was associated with a 2.5 greater likelihood for the prevalence of CAC. However, after accounting for age, this relationship was no longer evident (Table 2). Correlates of the prevalence of CAC were thus age and serum creatinine in stepwise multivariable analysis allowing for skin intrinsic fluorescence and other previously identified univariate correlates. In Table 2, N/A=not applicable; N/S=not selected; * signifies Model 4 Stepwise selection model allowed for the following variables: skin intrinsic fluorescence, age, sex, coronary artery disease, HbA1c, BMI, natural logarithmically transformed serum creatinine, natural logarithmically transformed albumin excretion rate, high density lipoprotein cholesterol, non-high density lipoprotein cholesterol, diastolic blood pressure, use of hypertension medication, use of statins, and a history of smoking.

TABLE 2

| | Model 1 | Model 2 | Model 3 | Model 4* |
|---|---|---|---|---|
| Skin Intrinsic | 2.51 | N/S | N/S | N/S |
| Fluorescence | (1.37-4.59) | | | |
| Age (years) | N/A | 3.43 | 3.43 | 3.11 |
| | | (1.74-6.76) | (1.74-6.76) | (1.57-6.16) |
| (log) Serum | N/A | N/A | N/S | 2.71 |
| creatinine | | | | (1.07-6.87) |
| (mg/dl) | | | | |

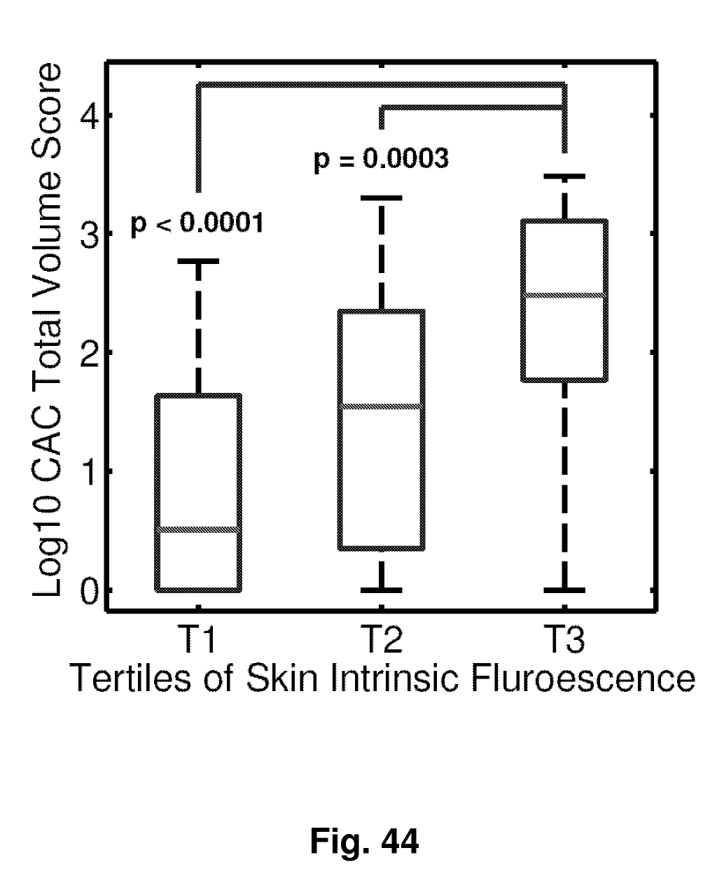

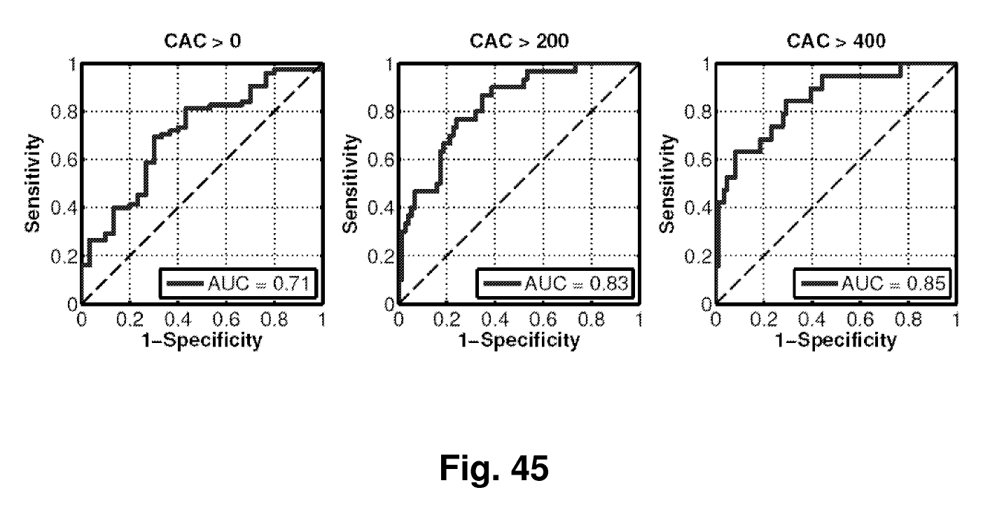

FIG. 44 demonstrates the median (log 10) CAC score by tertiles of skin intrinsic fluorescence. There was a marked increase in the severity of CAC with each increasing tertile of skin intrinsic fluorescence. FIG. 45 shows the discriminative ability to detect CAC at threshold scores of >0, >200, and >400, representing 71, 30, and 19% of the population, respectively. Although skin intrinsic fluorescence shows minimal ability to detect the presence of any CAC, its discriminative ability increases with increasing threshold scores of total CAC. The area under the curve for the presence of CAC (a CAC score >0) is 71%. This increases to 82% at a threshold score of >200 and to 85% at a threshold score of >400.

When looking at the severity of CAC, i.e. CAC score, skin intrinsic fluorescence demonstrated a strong association with the most recent CAC score (r=0.54, p<0.0001), which remained even after adjusting for age (r=0.38, p<0.0001). In multivariable analysis allowing for age, sex, HbA1c, BMI, serum creatinine (renal function), albumin excretion rate (renal damage), HDL cholesterol, non-HDL cholesterol, diastolic blood pressure, hypertension medication use, and a history of smoking, skin intrinsic fluorescence still remained independently associated with the severity of CAC, i.e. the most recent CAC score (Table 3, Correlates of the Severity of Coronary Artery Calcification (CAC) (total volume score), β±SE (p-value)). Other independent correlates were age, CAD, albumin excretion rate, and a history of smoking. In Table 3, Model 4 Stepwise selection model allowed for the following variables: skin intrinsic fluorescence, age, sex, coronary artery disease, HbA1c, BMI, natural logarithmically transformed serum creatinine, natural logarithmically transformed albumin excretion rate, high density lipoprotein cholesterol, non-high density lipoprotein cholesterol, diastolic blood pressure, use of hypertension medication, use of statins, and a history of smoking.

TABLE 3

| | Model 1 | Model 2 | Model 3 | Model 4 |
|---|---|---|---|---|
| Skin Intrinsic | 1.45 ± 0.22 | 1.01 ± 0.24 | 0.89 ± 0.23 | 0.56 ± 0.26 |
| Fluorescence | (<0.0001) | (<0.0001) | (0.0002) | (0.03) |
| Age (years) | | 0.92 ± 0.25 | 0.79 ± 0.25 | 0.97 ± 0.25 |
| | | (0.0004) | (0.002) | (0.0002) |
| Total CAD | | | 1.36 ± 0.49 | 1.08 ± 0.49 |
| | | | (0.006) | (0.03) |
| BMI (kg/m²) | | | | 0.34 ± 0.21 |
| | | | | (0.11) |
| (log) AER | | | | 0.48 ± 0.24 |
| (μg/min) | | | | (0.05) |
| A history of | | | | 0.83 ± 0.45 |
| smoking | | | | (0.07) |

In subanalyses of the eighty participants who had previously undergone EBT scanning for CAC in 1996-1998 (CAC baseline), correlates of the progression of CAC were investigated. Table 4 demonstrates the relationship of skin intrinsic fluorescence with the progression of CAC in the participants with any data on progression, those with a baseline CAC score of 0, and in those with a CAC score greater than zero at baseline, respectively. Skin intrinsic fluorescence was significantly associated with CAC progression (OR=2.2, 1.09-4.45), even after allowing for age and baseline CAC score, serum creatinine, albumin excretion rate, and other univariately or clinically significant correlates. Final multivariable correlates of CAC progression were skin intrinsic fluorescence and baseline CAC score. In those with a baseline CAC score=0, skin intrinsic fluorescence was the only variable selected in the stepwise selection model, demonstrating a marginal association with CAC progression (p=0.08). In those with a CAC score >0 at baseline, skin intrinsic fluorescence was neither univariately nor multivariately associated with CAC progression. In the final multivariable model, only the baseline age was associated with the progression of CAC in those with a baseline CAC score greater >0.

Coronary Artery Calcification

Putative mechanisms of vascular calcification include calcium deposition into the arterial wall as a result of increased parathyroid hormone activity and elevated extraosseous calcium and phosphorous levels, as observed in kidney disease; vascular smooth muscle cell and calcifying vascular cell differentiation into osteoblastic cells; macrophage ingestion of elevated oxidized low density lipoprotein cholesterol levels which induces vascular smooth muscle cell migration from the media to intima layer and secretion of collagen fibers that trap calcium and apatite crystals; and effects of advanced glycation end products indirectly via low density lipoprotein cholesterol or directly by inducing osteblastic differentiation of pericytes/vascular smooth muscle cells. Although parathyroid hormone, serum calcium or phosphorous levels were not measured and whether the relationship of skin intrinsic fluorescence was independent of or interacted with that of calcium and phosphorous could not be determined in the study, lipids, i.e. high density lipoprotein cholesterol or non-high density lipoprotein cholesterol, did not demonstrate a relationship with CAC nor did calculated low density lipoprotein cholesterol in the 70% of participants with fasting data. Nevertheless, skin intrinsic fluorescence, a marker of AGEs, was associated with the presence (univariately), and severity of CAC, and demonstrated an association in detecting those who had shown a progression in CAC. While an association of lipids with CAC in this population could not be discounted due to the cross-sectional nature of the study design and limited sample size, a relationship of non-high density lipoprotein cholesterol with progression has indeed been previously shown and these results do suggest that factors other than the traditional atherosclerotic risk factors influence the CAC observed in this population. See T. Costacou et al., “Progression of Coronary Artery Calcification in Type 1 Diabetes Mellitus,” Am J Cardiol 100:1543-7, 2007.

AGEs have been shown to induce vascular calcification and to upregulate mRNAs coding for markers of early and late phase osteoblastic differentiation. Yamagishi et al. demonstrated that AGEs upregulated osteoblastic differentiation of vascular pericytes. See S. Yamagishi et al., “Advanced glycation end products accelerate calcification in microvascular pericytes,” Biochemical and Biophysical Communications 258:353-7, 1999. A direct relationship between measures of abdominal adiposity and BMI and the prevalence of CAC, but inverse relationships between these body fat indices and the severity of CAC, in those with CAC, have been previously observed in this population. This might be due to EBT detecting CAC due to both lipid accumulation and hypertension induced smooth muscle cell proliferation of the intima wall on the one hand and AGE-induced calcification of the medial wall on the other hand. The results of the study support this. After controlling for skin intrinsic fluorescence, BMI showed a marginally direct association with the severity of CAC (in the model not adjusted for coronary artery disease-data not depicted). Skin intrinsic fluorescence was also associated with CAC severity, suggesting that both provided independent information on the extent of calcification of the coronary arteries.

In a previous report from the T1D population, a strong relationship between radiographically determined medial wall calcification of the ankle and EBT determined calcification of the coronary arteries was shown. See T. Costacou et al., “Lower-extremity arterial calcification as a correlate of coronary artery calcification,” Metabolism 55:1689-96, 2006. Sakata et al. observed higher levels of calcification of the medial wall of the internal thoracic artery in diabetic patients. See N. Sakata et al., “Calcification of the Medial Layer of the Internal Thoracic Artery in Diabetic Patients: Relevance of Glycoxidation,” J Vasc Res 40:467-574, 2003. In their population, internal thoracic calcification was associated with AGEs in those with diabetes, but not in those without diabetes.

While it is possible that the association between skin intrinsic fluorescence and CAC is due to confounding by renal damage, as both increase with declining renal function and increasing renal damage, the association with the severity of CAC was independent of renal function, i.e. serum creatinine, although not of renal damage, i.e. albumin excretion rate. In hemodialysis patients, independent of duration on dialysis, the fluorescent AGE, pentosidine, was associated with CAC. See K. Taki et al., “Oxidative stress, advanced glycation end product, and coronary artery calcification in hemodialysis patients,” Kidney Int 70:218-24, 2006.

CAC is a measure of atherosclerotic burden and medial wall CAC is associated with atherosclerotic disease. Rumberger et al. were able to use EBT determined calcification to discriminate between 50% stenosis and no obstructive disease, but not the extent of stenosis in one hundred and thirty-nine men and women. See J. Rumberger et al., “Coronary Calcium, as Determined by Electron Beam Computed Tomography, and Coronary Disease on Arteriogram,” Circulation 91:1363-7, 1995. Higher levels of the soluble receptor for AGEs were cross-sectionally associated with cardiovascular disease in individuals with type 1 diabetes in the EURODIAB study. See J. Nin et al., “Levels of soluble receptor for AGE are cross-sectionally associated with cardiovascular disease in type 1 diabetes, and this association is partially mediated by endothelial and renal dysfunction and by low-grade inflammation: the EURODIAB Prospective Complications Study,” Diabetologia 52:705-14, 2009. Skin autofluorescence, as detected by the AGEReader™ (DiagnOptics BV, Groningen, the Netherlands), has very recently been shown to predict cardiovascular events in a cohort of 973 subjects with type 2 diabetes. See H. Lutgers et al., “Skin autofluorescence provides additional information to the UK Prospective Diabetes Study (UKPDS) risk score for the estimation of cardiovascular prognosis in type 2 diabetes mellitus,” Diabetologia 52:789-97, 2009.

The study is limited by the cross-sectional nature of the study design. The calcification data were collected prior to measurement of skin intrinsic fluorescence, therefore these results show only an association at best. However, as the major outcome in this study was coronary artery calcification, which also shows a strong relationship with all-cause mortality, this cross-sectional association in living participants still has important implications. See L. Niskanen et al., “Medial artery calcification predicts cardiovascular mortality in patients with NIDDM,” Diabetes Care 17:1252-6, 1994.

The method of the present invention uses skin intrinsic fluorescence. Although the SCOUT DM instrument has the unique ability to measure both skin autofluorescence and intrinsic fluorescence, intrinsic fluorescence has been found by the inventors to be a more reliable measure of fluorescence across varying skin types and pigmentation because it compensates for optical absorption by hemoglobin and melanin in the emission region, whereas autofluorescence does not.

Skin intrinsic fluorescence shows a cross-sectional association with coronary artery calcification and recent progression of coronary artery calcification in type 1 diabetes. The relationship of this spectroscopically determined marker of advanced glycation end products and coronary artery calcification appears stronger with more severe calcification. Given the strong relationship of coronary artery calcification with coronary artery disease and its even stronger relationship with mortality, the finding of a relationship of skin intrinsic fluorescence with coronary artery calcification, independent of age, a history of coronary artery disease, renal function, or renal damage, has important implications. To what degree skin intrinsic fluorescence and AGE formation are truly causative in the pathways to CAC and CAD cannot be determined by observational data such as these alone. However, these data suggest that skin intrinsic fluorescence can be a useful marker of CAC/CAD risk and can be useful as a potential a therapeutic target.

A study comparing skin intrinsic fluorescence measured by SCOUT DM to CAC measured with a 64-slice, rapid computed tomography scanner in 155 subjects largely without diabetes (N=143) was conducted at the New Mexico Heart Institute (NMHI) in 2009. The sum of the skin intrinsic fluorescence excited with a 460 nm LED and detected over the 490 to 660 nm spectral range was computed by first correcting the measured fluorescence using the intrinsic fluorescence correction equation previously described with Kx=1.0 and Km=0.2 and then taking the sum of the individual wavelengths of the intrinsically corrected fluorescence. Finally the intrinsic fluorescence sum was multiplied by 1000. A summary of the cohort demographics is provided in Table 4.

TABLE 4

| | N +/− | +Mean (Std) | −Mean (Std) | R | P-Val |
|---|---|---|---|---|---|
| SIF (AU) | 51/103 | 1.54 (0.55) | 1.25 (0.29) | 0.39 | 2.6E−07 |
| Age (CACS) | 51/105 | 64.2 (8.9) | 60.5 (7.8) | 0.30 | 0.008 |
| Gender (CACS) | 65/90 | 147.6 (287.2) | 547.6 (1362.5) | | 0.021 |
| Diabetes (CACS) | 12/143 | 1109.6 (2827.1) | 318.6 (758.5) | | 0.012 |
| BMI (kg/m²) | 51/104 | 27.9 (5.7) | 27.8 (5.2) | −0.03 | 0.87 |
| Waist (inches) | 51/104 | 37.4 (9.8) | 35.6 (10.3) | 0.05 | 0.32 |
| Smoking current (CACS) | 7/146 | 294.6 (115.1) | 378.5 (1100.1) | | 0.84 |
| Smoking never (CACS) | 73/80 | 320.9 (661.1) | 423.8 (1351.5) | | 0.56 |
| Hypertension (CACS) | 66/88 | 379.7 (1266.2) | 372.9 (910.8) | | 0.97 |
| Dyslipidemia (CACS) | 92/62 | 342.3 (846.3) | 425.5 (1350.1) | | 0.64 |

As shown in Table 4, skin intrinsic fluorescence (SIF) was statistically significantly higher (p=2.6e-7) in the 51 subjects who had CAC scores 200 (+), than the 103 subjects who had CAC scores <200. In this cohort, there were 65 women (one woman had diabetes) and 90 men (11 men had diabetes). Males had higher CAC scores than females. In addition, older subjects had higher CAC than younger subjects. Finally, the 12 patients with diabetes (11 with type 2 and 1 with type 1) had significantly higher CAC than the 143 patients without diabetes. Coronary artery disease risk factors such as elevated body mass index (BMI), elevated waist circumference, smoking currently or ever, hypertension and dyslipidemia were not significant in their association with CAC≧200.

The correlation of the SIF sum with CAC, natural logarithm of CAC, log 10 of CAC or the square root of CAC can be improved by adjusting the SIF sum with a multivariate models such as logistic regression or linear regression that account for factors that can affect skin fluorescence such as the age of the subject, gender, ethnicity, diagnosed type 1 or type 2 diabetes, skin tone (as quantified by the sum of the skin reflectance across the fluorescence emission band), smoking (i.e. current, previous vs never smoker, pack years or current smoker times duration of smoking), renal function (estimated glomerular filtration rate, albumin excretion ratio), waist circumference, waist-to-hip ratio, BMI, systolic and/or diastolic blood pressure, use of blood pressure medication, diagnosis of hypertension, lipid levels, use of cholesterol medication (e.g. statins), fasting glucose concentration, glycosylated hemoglobin concentration (HbA1c), casual glucose concentration, glucose concentration in response to a glucose challenge test, fructosamine, 1,5-anhydro-D-glucitol, c-reactive protein, other markers of inflammation or markers of oxidative stress (e.g. isoprostances).

Table 5 is an illustrative example of improved correlation (R) between square root of CAC and a linear regression model based on the SIF sum and various combinations of age (years), gender (1=male, 2=female), diagnosed diabetes (0=no diabetes, 1=diagnosed diabetes), ethnicity(0=white or Hispanic, 1=all other ethnicities), skin tone (sum of skin reflectance over fluorescence emission band) and smoking status (0=never smoked, 1=current or previous smoker). The model produced by the linear regression contains a constant (b₀) and weights (b₁-b_{N}) for the N variables in the model. For example, in a model that utilizes SIF sums, age, gender, diabetes status, skin tone, ethnicity and smoking status, the square root of CAC is calculated with the following formula:

Sqrt(CAC)=b₀+b₁*ΣSIF+b₂*Age+b₃*Gender+b₄*Diabetes+b₅*Skin Tone+b₆*Smoking+b₇*Smoking

The use of the additional information with the SIF sum improves the correlation when considering the entire cohort (All), men only and women only. The correlation with the entire cohort improves by as much as 24% and in men by as much as 29%.

TABLE 5
Linear Regression Model Correlation with Square Root of CAC

| Model Variables | All | Men | Women |
|---|---|---|---|
| ΣSIF | | | |
| ΣSIF + Age | .451 | .435 | .574 |
| ΣSIF + Age + Gender | .485 | .435 | .574 |
| ΣSIF + Age + Gender + Diabetes | .528 | .536 | .576 |
| ΣSIF + Age + Gender + Diabetes + Skin Tone | .531 | .537 | .582 |
| ΣSIF + Age + Gender + Diabetes + | .531 | .541 | .584 |
| Skin Tone + Smoking | | | |

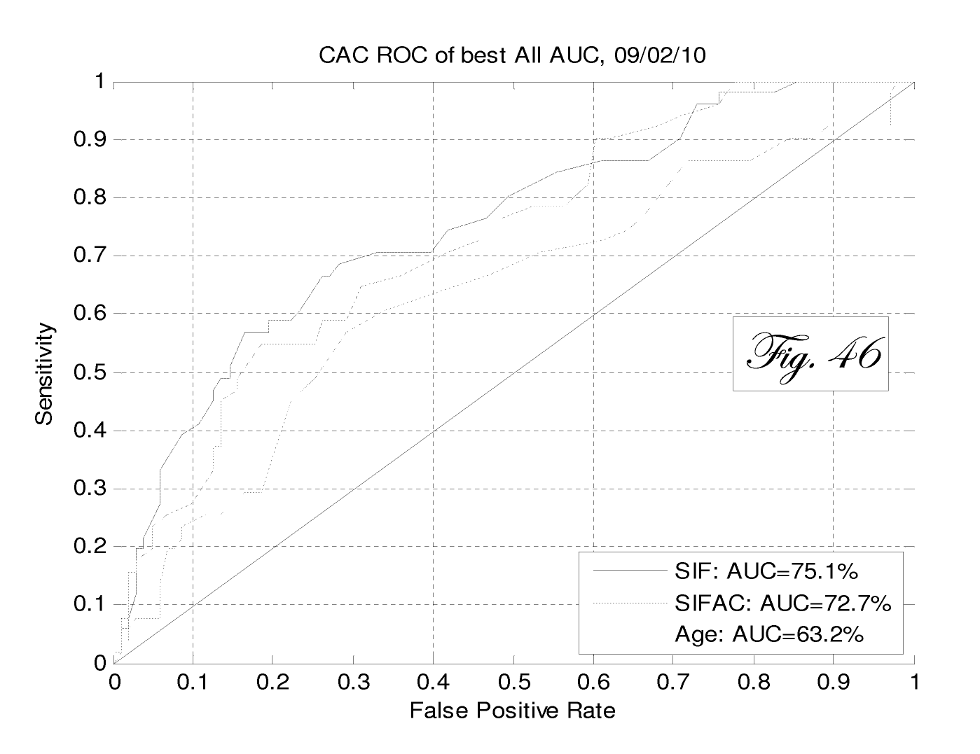

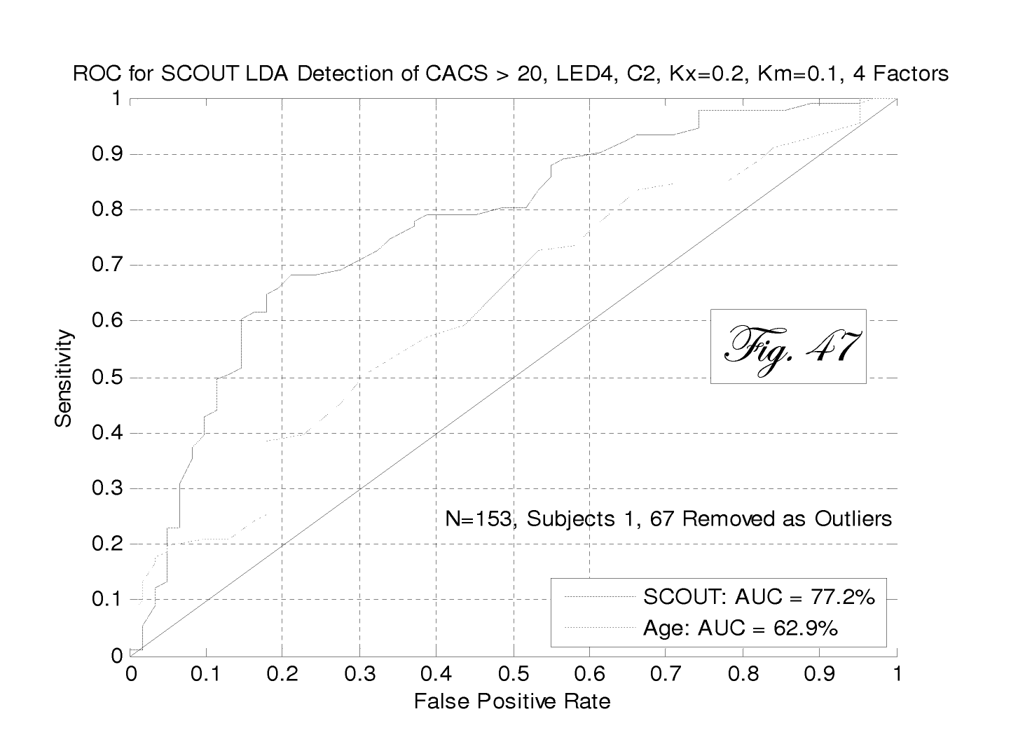

FIG. 46 is a graph of the receiver operator characteristic (ROC) for the detection of CAC≧200 in the NMHI cohort using the SIF sum of each participant. The SIF sum has good detection ability with an area under the curve (AUC) of 75.1% and sensitivity of nearly 70% at a 30% false positive rate (FPR). While the SIF sum works well, it does not take advantage of spectral shape information present in the intrinsically corrected fluorescence which can add further discriminative power. To determine if spectral shape improved detection of CAC, multivariate linear discriminate analysis was applied to the SIF spectra from the NMHI cohort. Firstly, the fluorescence spectra generated by an excitation LED centered at 435 nm and detected over the 460 to 660 nm emission window were intrinsically corrected using the previously described intrinsic correction technique with kx=0.2 and km=0.1. The spectra were decomposed into orthogonal sources of spectral variance and corresponding scores using principal components analysis. The resulting scores were supplied to the linear discriminant analysis algorithm to find the hyper-plane that best separated subjects with CAC<20 (no significant CAC) from those with CAC≧20. The CAC≧20 threshold is a more difficult test and as shown in the ROC of FIG. 47, the AUC for detection of CAC≧20 is 77.2% with a sensitivity of 70% at a 30% FPR. This is significantly better than what was achieved with simple fluorescence sums.

In addition to using spectral shape for qualitative detection of CAC above a given threshold, quantitative multivariate models can be constructed that relate the spectral shape to the CAC level in a given subject. Example multivariate algorithms that can be employed include multiple linear regression, partial least squares regression, principal components regression, multivariate adaptive regression splines (MARS), generalized linear models (GLM) and support vector regression (SVR). These type of models can be built for use on all subjects and/or specific subgroups such as men vs women, ethnic groups, age tertiles or other subgroups as suggested by the data. Finally, multiple multivariate models can be combined through a technique called ensembling to produce even more accurate results. Simple ensembling techniques include averaging the outputs of the individual models and voting based on consistency of the individual model outputs. More sophisticated ensembling techniques utilize linear regression on the individual model outputs to elucidate an optimal weighting (i.e. unequal contribution) of each individual model output to form the final output.

The multivariate models built from the spectral measurement can be extended leading to improved accuracy by appending certain information to the spectra before applying the multivariate model. The appended information can include any combination or subset of the following including the age of the subject, gender, ethnicity, smoking (i.e. current, previous vs never smoker, pack years or current smoker times duration of smoking), renal function (estimated glomerular filtration rate, albumin excretion ratio), waist circumference, waist-to-hip ratio, BMI, systolic and/or diastolic blood pressure, use of blood pressure medication, diagnosis of hypertension, lipid levels, use of cholesterol medication (e.g. statins), fasting glucose concentration, glycosylated hemoglobin concentration (HbA1c), casual glucose concentration, glucose concentration in response to a glucose challenge test, fructosamine, 1,5-anhydro-D-glucitol, c-reactive protein, other markers of inflammation or markers of oxidative stress (e.g. isoprostances). The resulting feature vector can yield improved performance relative to just the spectral information because it contains more information useful for determining CAC levels.

The detection of CAC in a subject with skin intrinsic fluorescence (SIF) has several potential uses including using the SIF measurement to screen for CAC in individuals who do not have symptoms of coronary artery disease, to screen for levels of CAC that connote increased risk of CAD and/or future heart attack (e.g. myocardial infarction) to identify individuals who are asymptomatic for CAD but should be sent to have CAC measured by EBT or multi-slice rapid computed tomography or as a direct indicator of CAD and/or cardiovascular disease risk. In addition, the SIF measurement can be used to reclassify subjects with low or intermediate risk for heart disease as assessed by the Framingham risk equation. For example, if a subject has intermediate Framingham risk but a high SIF measurement of CAC, that subject would be reclassified as having high Framingham risk and his/her medical treatment adjusted appropriately. Likewise, if a subject has intermediate Framingham risk but a low SIF measurement of CAC, that subject would be reclassified as having low Framingham risk and medical treatment for CVD would be reduced. Finally, if a subject had low Framingham risk and a high SIF measurement of CAC, that subject would be reclassified as having intermediate Framingham risk for CVD and his/her medical treatment and monitoring might be increased.

The SIF measurement has safety, convenience and cost saving advantages over measuring CAC. The SIF measurement is safer than a CAC measurement because it uses harmless, non-ionizing radiation to detect changes in the skin related to CAC accumulation and since most subjects do not have significant CAC, this reduces unnecessary exposure to radiation. The SCOUT instrument for measuring SIF is portable and relatively inexpensive facilitating deployment at clinics, health fairs, employee wellness clinics, pharmacies and doctors' offices. The measurement takes less than 5 minutes to perform and can be done opportunistically at the point of service. This convenience factor facilitates screening many more individuals for CAC, CAD and CVD than can be done with EBT or multi-slice rapid CT which require a dedicated facility and radiologist or cardiologist to interpret the results. Finally, the SIF measurement is an order of magnitude less expensive to perform than EBT or multi-slice rapid CT.

Improved Instrumentation for Noninvasive Detection of Disease

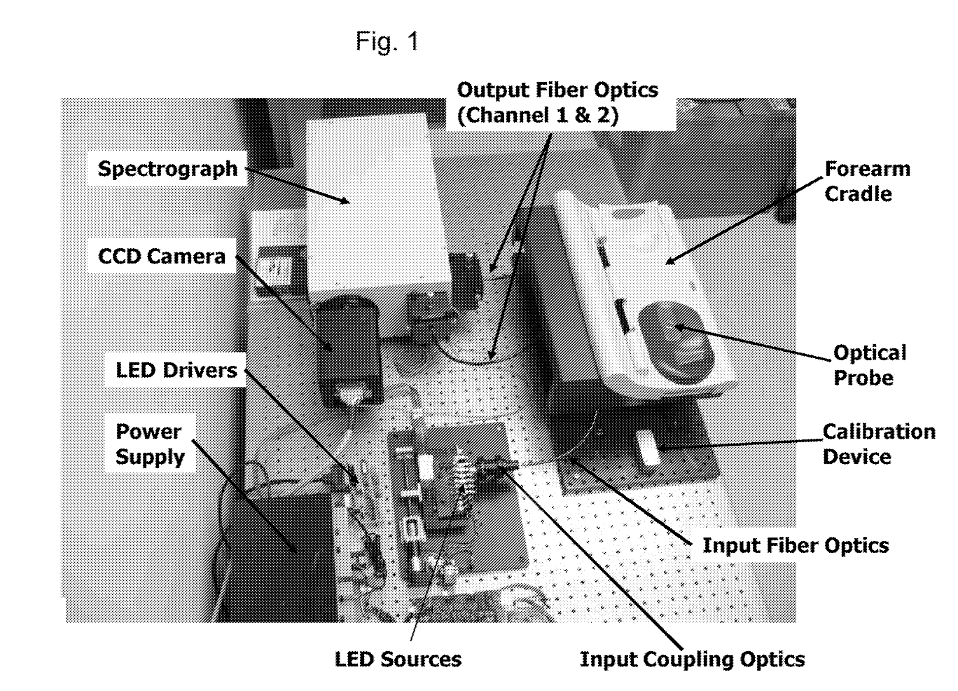

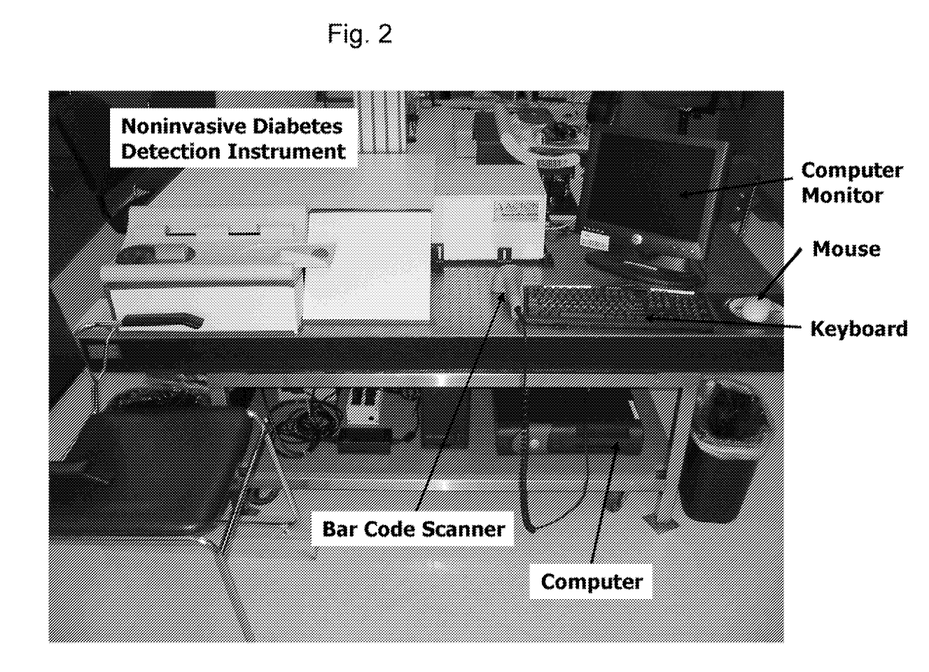

A spectroscopic apparatus that can be used with the present invention can comprise an instrument specifically designed to use fluorescence and reflectance spectroscopy to noninvasively detect disease in an individual. FIG. 1 and FIG. 2 depict a representative embodiment of such an instrument and its major subsystems. Generally, the system includes a light source, an optical probe to couple light from the light source to an individual's tissue and to collect reflected and emitted light from the tissue, a forearm cradle to hold a subject's arm still during the optical measurement, a calibration device to place on the optical probe when instrument calibration is required, a spectrograph to disperse the collected light from the optical probe into a range of wavelengths, a CCD camera detection system that measures the dispersed light from the tissue, a power supply, a computer that stores and processes the CCD camera images plus controls the overall instrument and a user interface that reports on the operation of the instrument and the results of the noninvasive measurement. Additional descriptions of suitable apparatuses can be found in U.S. application Ser. No. 11/964,675, incorporated herein by reference.

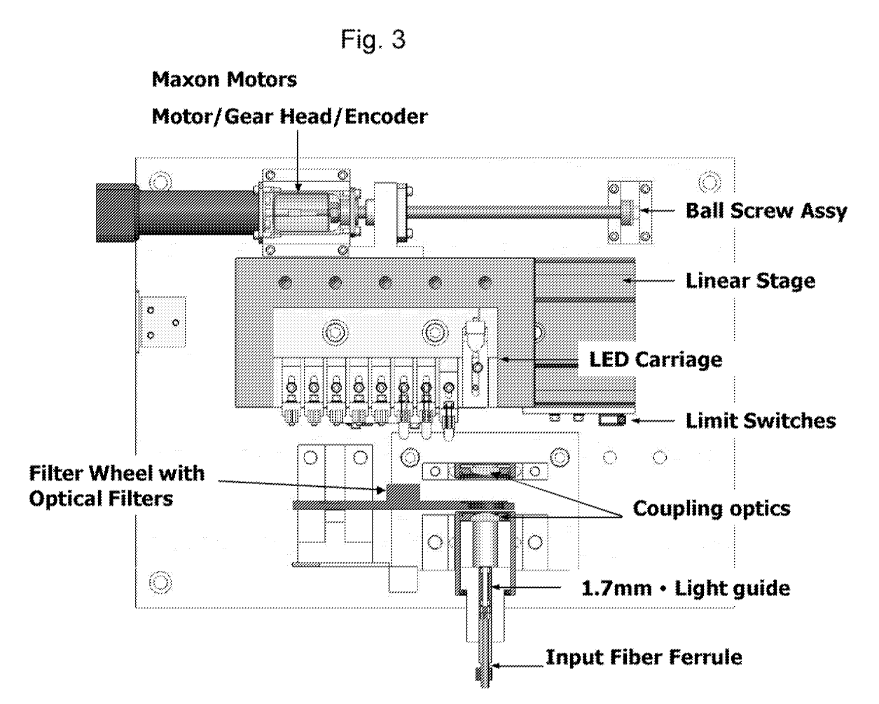

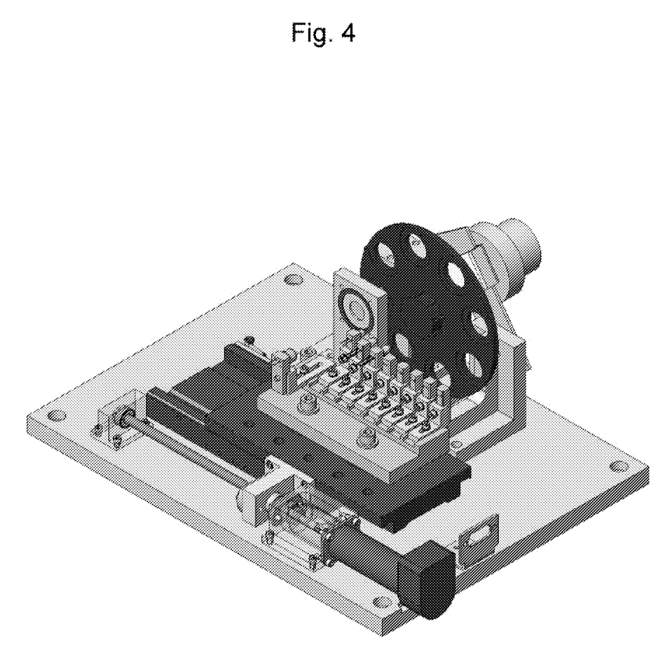

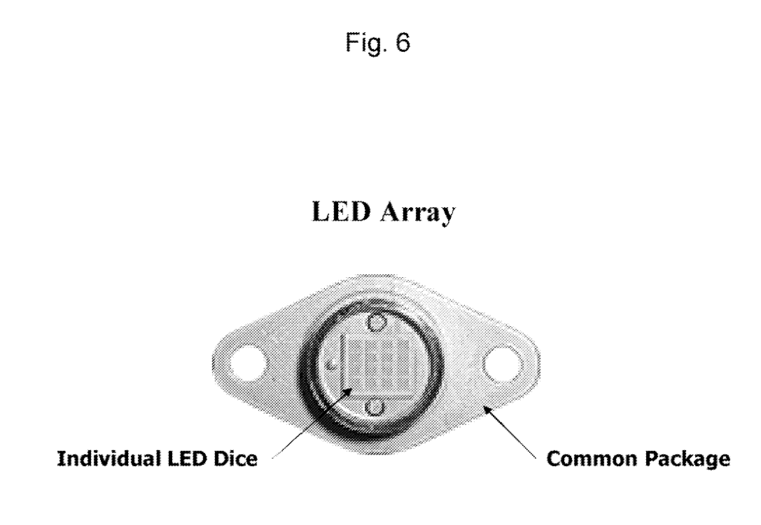

The light source subsystem can utilize one or more light emitting diodes (LEDs) to provide the excitation light needed for the fluorescence and reflectance spectral measurements. The LEDs can be discrete devices as depicted in FIG. 3 or combined into a multi-chip module as shown in FIG. 6. Alternately, laser diodes of the appropriate wavelength can be substituted for one or more of the LEDs. The LEDs emit light in the wavelength range of 265 to 850 nm. In a preferred embodiment of the SCOUT light source subsystem the LEDs have central wavelengths of 375 nm, 405 nm, 420 nm, 435 nm and 460 nm, plus a white light LED is also used to measure skin reflectance.

The use of LEDs to excite fluorescence in the tissue has some unique advantages for noninvasive detection of disease. The relatively broad output spectrum of a given LED may excite multiple fluorophores at once. Multivariate spectroscopy techniques (i.e. principle components analysis, partial least squares regression, support vector regression, etc.) can extract the information contained in the composite fluorescence spectrum (i.e. a superposition of multiple fluorescence spectra from the excited fluorophores) to achieve better disease detection accuracy. The broad LED output spectrum effectively recreates portions of and excitation-emission map. Other advantages of using LEDs are very low cost, high brightness for improved signal to noise ratio, reduced measurement time, power efficiency and increased reliability due to the long lifetimes of the LED devices.

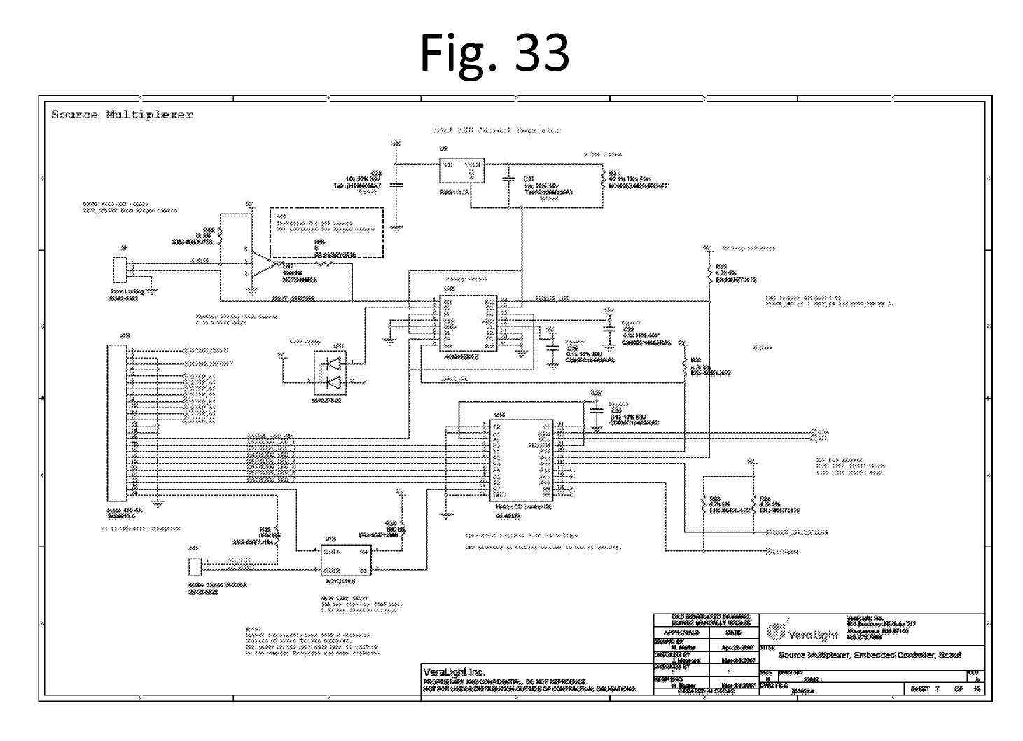

As shown in FIG. 3, the LEDs can be mechanically positioned in front of the coupling optics by a motor and translation stage. A LED driver circuit turns on/off the appropriate LED when it is positioned in front of the coupling optics. The LED driver circuit is a constant current source that is selectively applied to a given LED under computer control. An example LED driver circuit is shown in FIG. 33. This circuit includes a constant current source to drive the LEDs of the light source subsystem. The constant current source can be coupled to the anode of each light source LED and can be gated by a signal from the camera that indicates when an exposure is being taken. The cathode of each LED in the light source can be coupled to a programmable chip (U12) that selectively turns on a given LED by connecting the cathode to ground when commanded to do so. The LED can be turned on by the programmable chip (U12) in a continuous fashion or it can be turned on periodically using techniques such as pulse width modulation to selectively dim the LED for a given camera exposure time. It can be suitable to operate an embodiment of the present invention such that the LEDs of the example light source subsystem are turned on in sequence for a measurement cycle. The output light of the chosen LED is collected by a lens that collimates the light and sends the collimated beam through a filter wheel.

The filter wheel contains one or more filters that spectrally limit the light from a given LED. The filters can be bandpass or short pass type filters. They can be useful to suppress LED light leakage into the fluorescence emission spectral region. The filter wheel can also have a position without a filter for use with the white light LED or to measure unfiltered LED reflectance. If laser diodes are used instead of LEDs, the filter wheel and filters can be eliminated because of narrow spectral bandwidth of the laser diode does not significantly interfere with the collection of the fluorescence emission spectra.

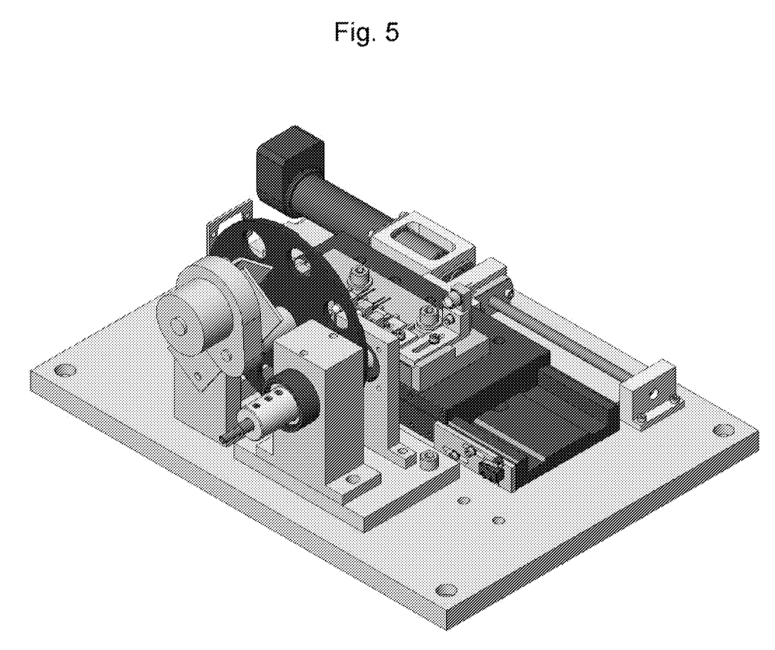

After light passes through the filter wheel, it is re-imaged by a second lens onto a light guide such as a square or rectangular light guide. The light guide scrambles the image from the LED and provides uniform illumination of the input fiber optic bundle of the optical probe. The optical probe input ferrule and the light guide can have a minimum spacing of 0.5 mm to eliminate optical fringing effects. The light guide can have at least a 5 to 1 length to width/height aspect ratio to provide adequate light scrambling and uniform illumination at the output end of the light guide. FIG. 4 and FIG. 5 show isometric views of an example light source subsystem.

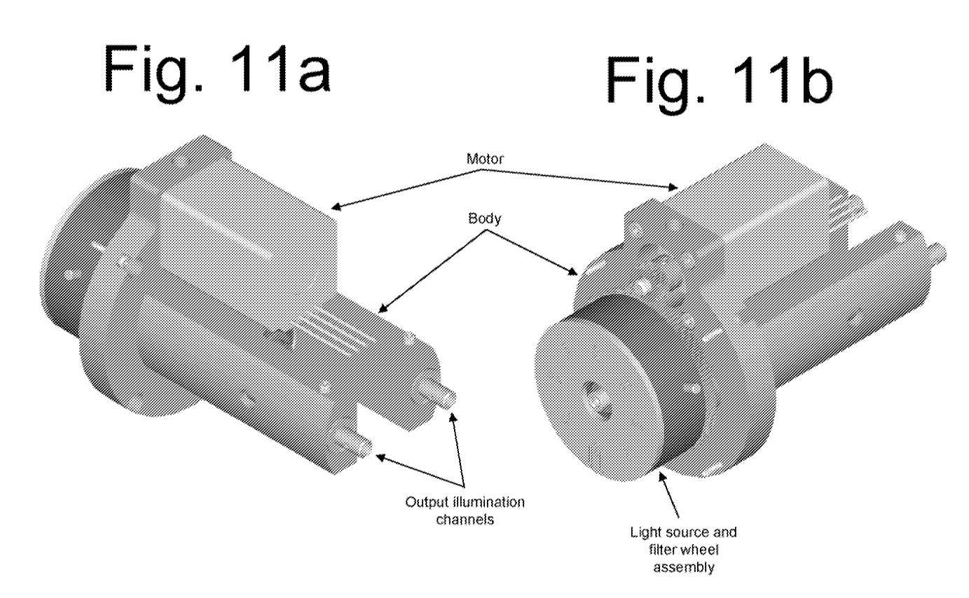

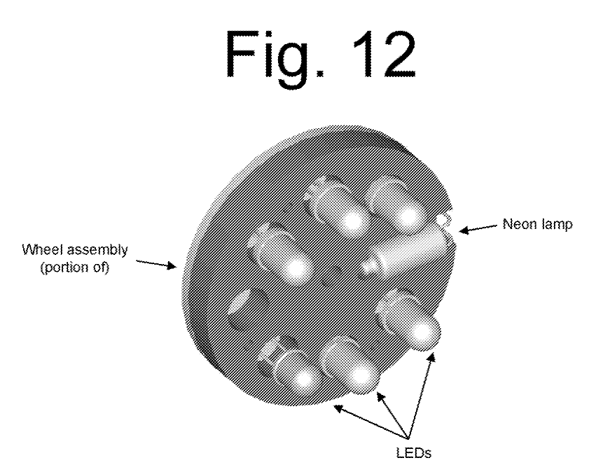

In an alternate embodiment of the light source subsystem, a plurality of illumination channels can be formed in order to accommodate the coupling of light into multiple fiber optic bundles of an optical probe. FIG. 11a and FIG. 11b depict front and back isometric views of an example embodiment having two output illumination channels. A main body provides support about which a wheel assembly, motor, coupling optics, and fiber optic ferrules are attached. The wheel assembly, a portion of which is shown in FIG. 12, is used to capture the LEDs, filters, and other light sources (e.g. a neon lamp for calibration). The wheel assembly attaches to a shaft that allows for the LED and filter assembly to rotate about a central axis. The attachment can be a direct coupling of the drive gear and the wheel gear, or a belt drive/linkage arrangement can be used. The belt drive arrangement requires less precision in the gear alignment and quiet operation (no gear grinding or vibration from misalignment). A motor is used to rotate the wheel assembly to bring the desired light source into alignment with the coupling optics that defines either of the two output illumination channels.

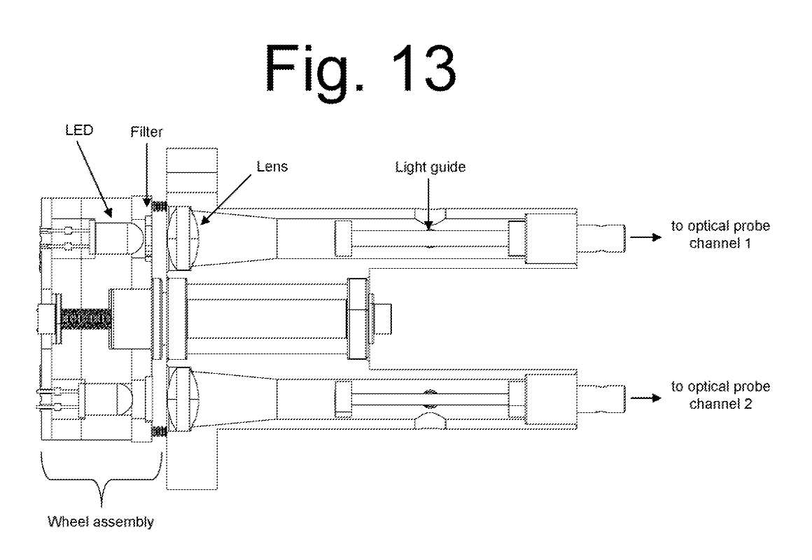

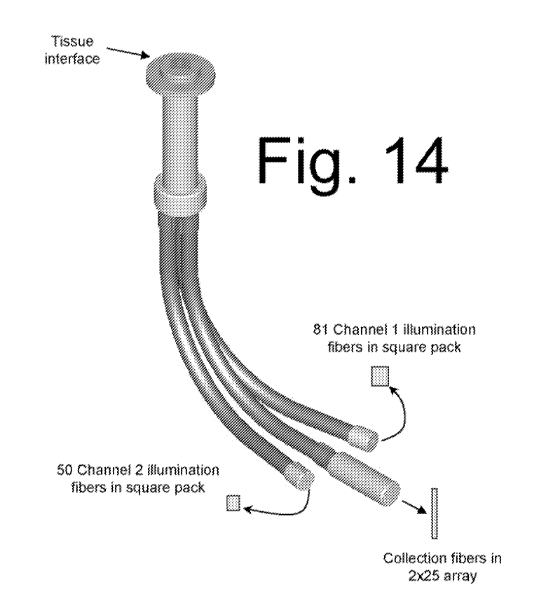

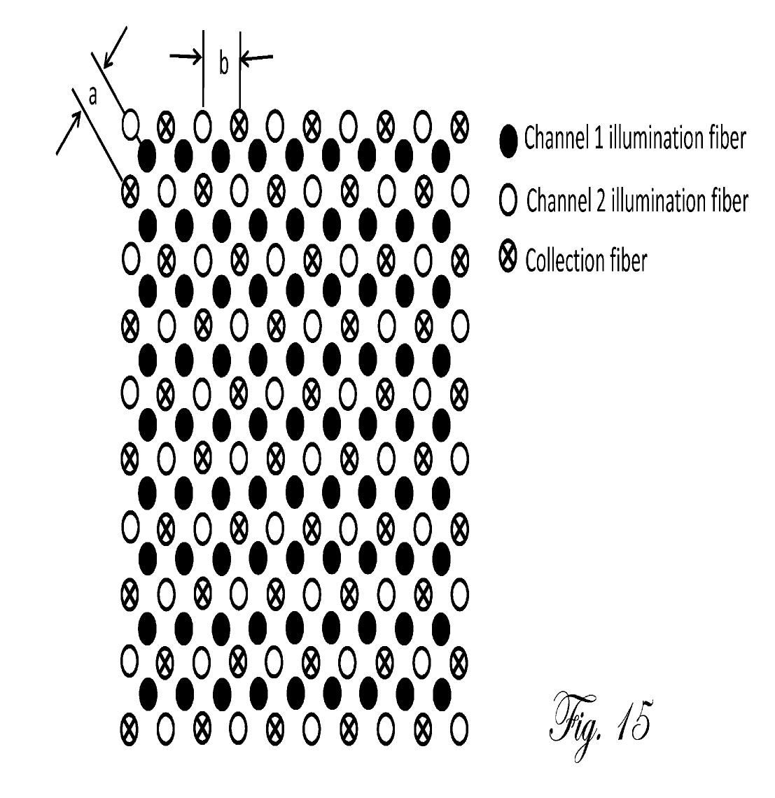

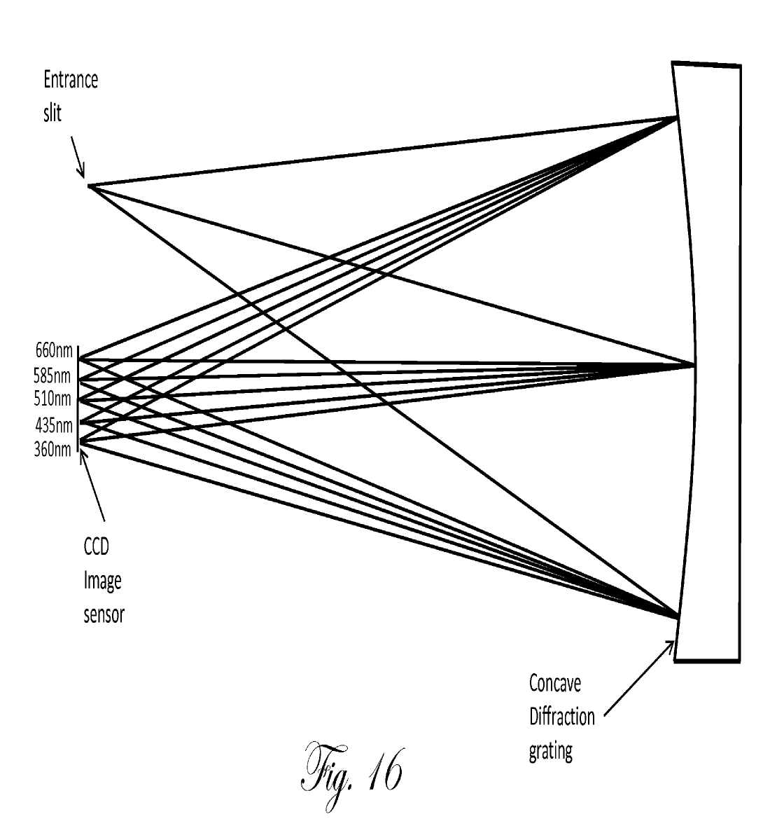

FIG. 13 shows a line drawing of a cross-sectional view of the light source subsystem through the two illumination channels. Considering only the upper most of the two channels, light is emitted by the LED and immediately passes through a filter. The light is then collected by a lens and re-imaged onto a light guide. The light guide homogenizes the spatial distribution of the light at the distal end, at which point it is butt-coupled to a corresponding fiber optic bundle of the optical probe. A second channel, shown below the first channel, is essentially a reproduction of the first, but has a light guide sized differently to accommodate a smaller fiber bundle.

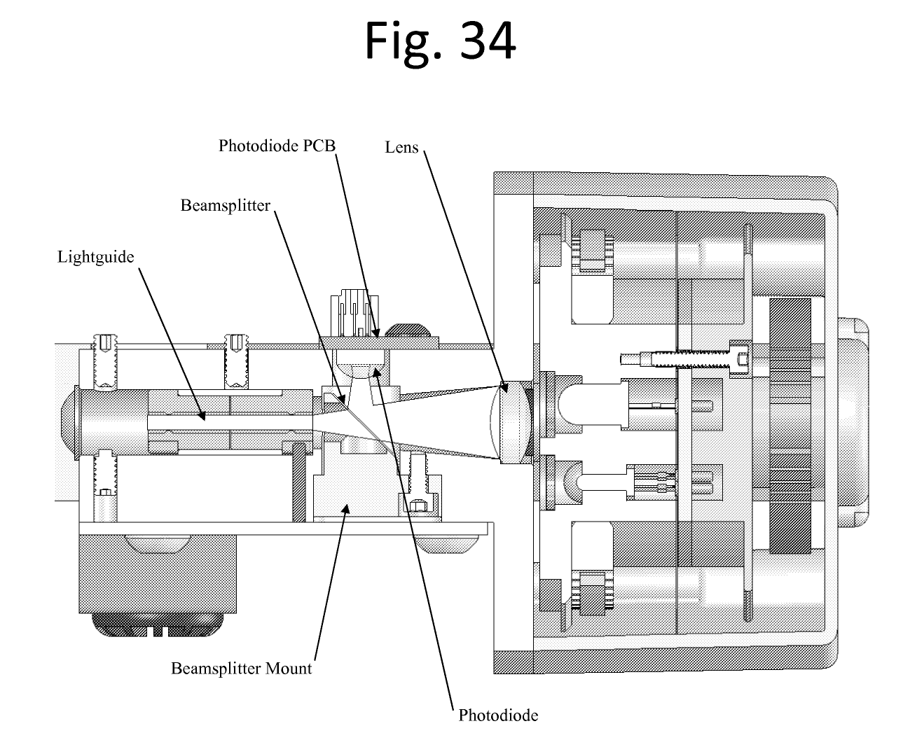

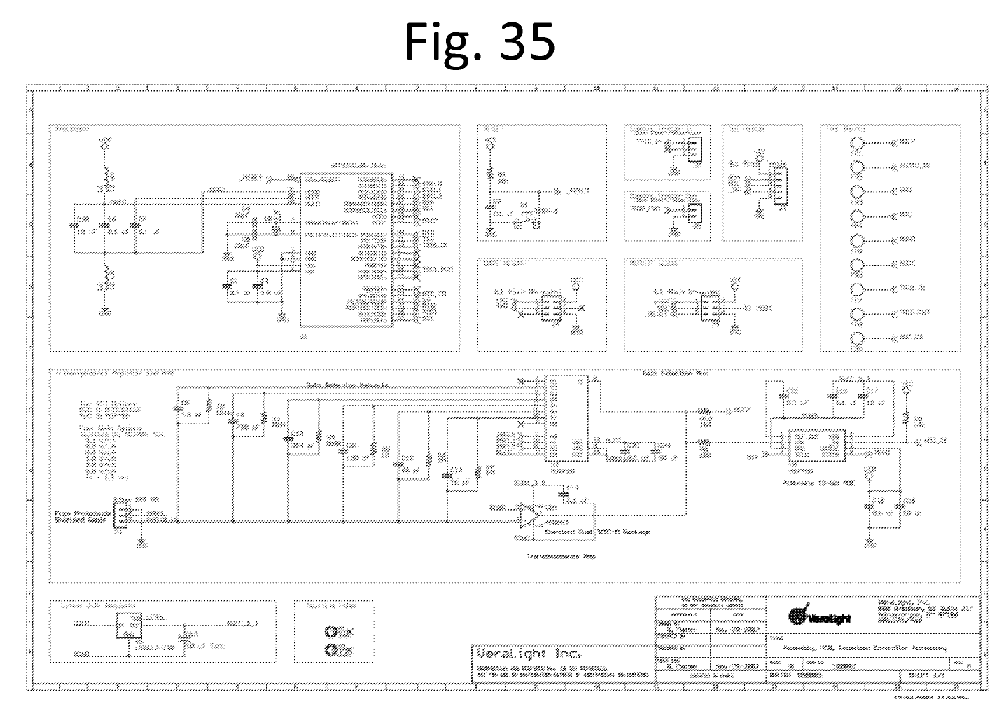

FIG. 34 shows another example embodiment of a light source subsystem. The example in FIG. 34 incorporates a mechanism to measure the intensity of the light shone on either input channel to allow compensation for LED output energy drifts due to changes over time and/or due to changes in device temperature induced by LED self-heating and/or ambient temperature. As shown in FIG. 34, a beamsplitter is placed in the optical path between the focusing lens and the light pipe. This is done in each input channel for the light source subsystem. The beamsplitter can be made of a material that is partially transmissive and partially reflective, such that some of the light is turned 90 degrees and directed onto a photodetector, while the remainder of the light passes through the beamsplitter and is directed onto a light guide or input of the optical probe. The photodetector converts the incident optical energy into a current that can be sensed with the circuit shown in FIG. 35. In FIG. 35, the current from the photodetector (sensitive to the wavelengths of light used for measurement of tissue state, etc.) is converted to a voltage by a transimpedance amplifier. The gain of the transimpedance amplifier can be fixed or programmable. In the example embodiment, the gain is chosen under computer control using an 8 to 1 analog multiplexer that selects the appropriate resistor/capacitor pair for the expected light level from the LED or light source. The output voltage of the transimpedance amplifier is coupled to an analog-to-digital converter (ADC) that digitizes the analog voltage into a code. The ADC resolution is application dependent, but typically ranges from 8 to 16 bits. In this particular embodiment, the ADC resolution is 12 bits. The ADC will digitize the output of the transimpedance amplifier upon command from the microcontroller in the circuit and transmits the digital output value to the microcontroller for use in quantifying the amount of light produced by the particular LED or light source that is shone onto the optical channel.

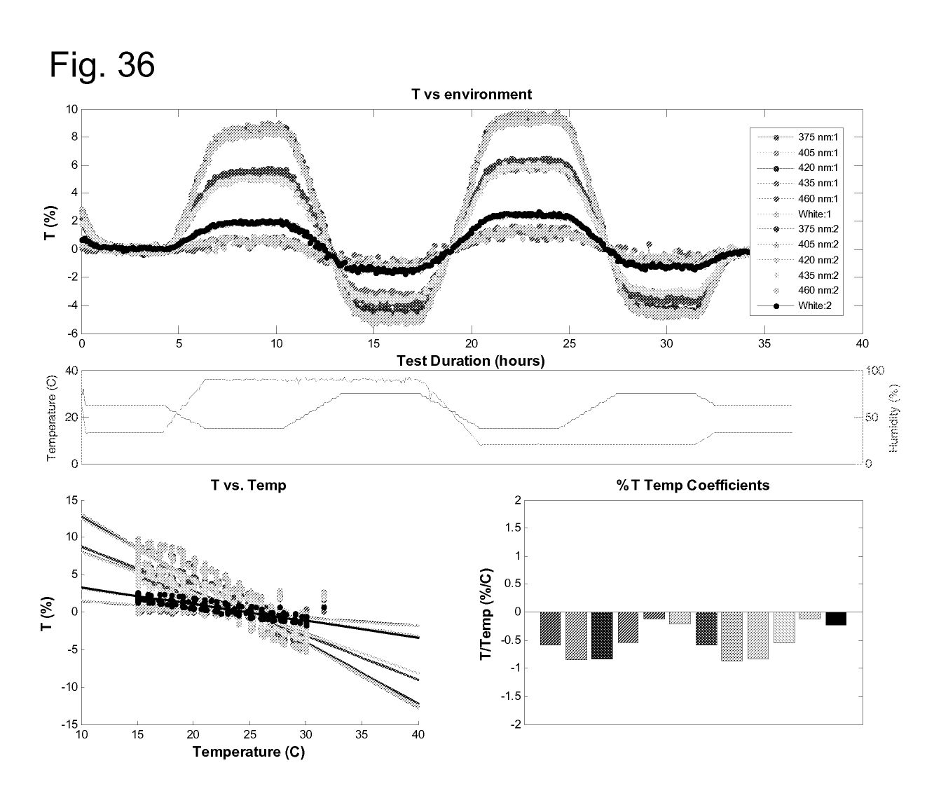

Quantifying the output of the light source can be useful for maintaining the calibration of the instrument and reducing the errors that can be produced due to drift in the LED output energy over time. FIG. 36 is an illustration of examples of the output energy drift of six different LEDs due to intentional perturbation of the ambient temperature. The upper graph of FIG. 36 shows the % change in transmission (% T) for LEDs with central wavelengths of 375 nm, 405 nm, 420 nm, 435 nm, 460 nm and white light. The % T change per degree Celsius is shown in the lower right graph and ranges anywhere from 0.3%/deg. C. to 1.3%/deg. C. LED output drift due to temperature changes can occur due to ambient temperature changes and/or self-heating when the LED is turned on. These changes are significant and must be compensated for if accurate measurements are to be maintained. The measurement of the LED output energy by the previously described circuit in combination with periodic or on demand (i.e. when a significant temperature change is detected) measurement of the calibration device allows compensation for the drift in LED energy. This can provide the added benefit of allowing detection of a fouled/damaged optical probe or calibration device because the relationship between the output of a given LED or light source and the energy reflected by the calibration device should be constant for a given instrument.

Alternately, LED temperature can be kept stable by mounting the LED die onto a thermally conductive surface that pulls away the heat generated by the LED when it has current flowing. In addition, the thermally conductive surface can be held a constant temperature by a thermo-electric cooler (e.g., a Peltier element) that has a temperature sensor and control circuit to maintain the LED or LEDs mounted on the thermally conductive surface at a fixed temperature to limit the amount of amplitude change. The techniques of measuring the light output of the LEDs can be combined with keeping the LEDs at a constant temperature to achieve even higher stability and maintenance of the instrument calibration.

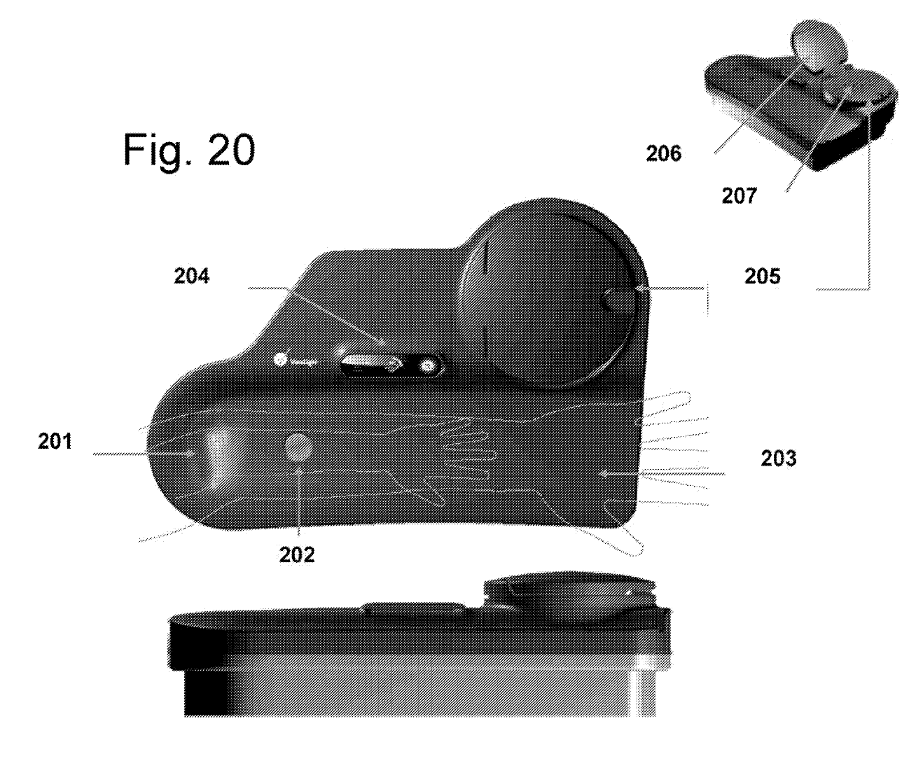

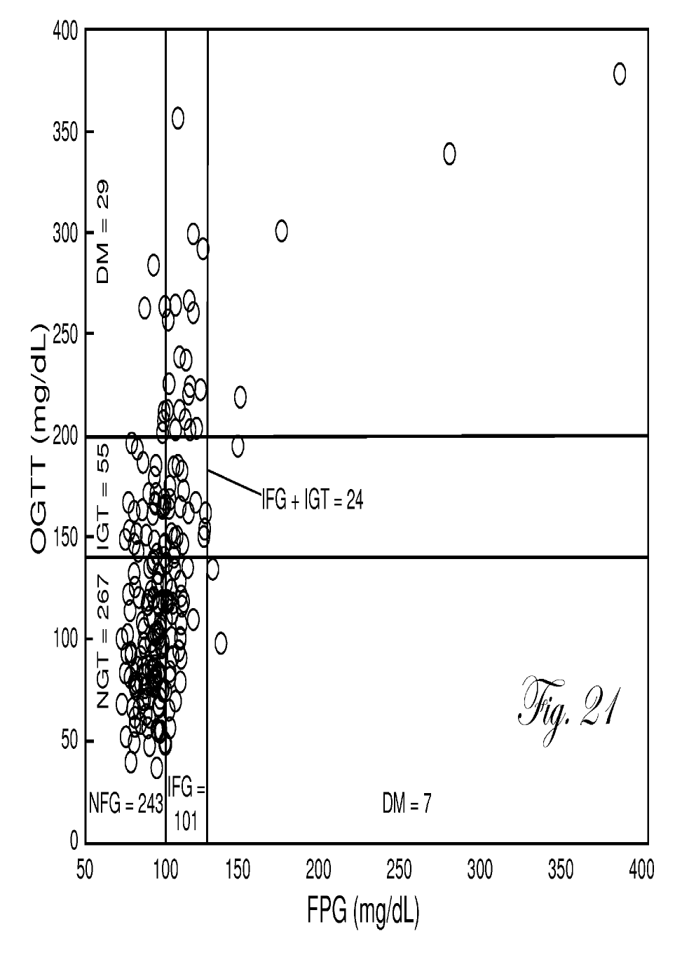

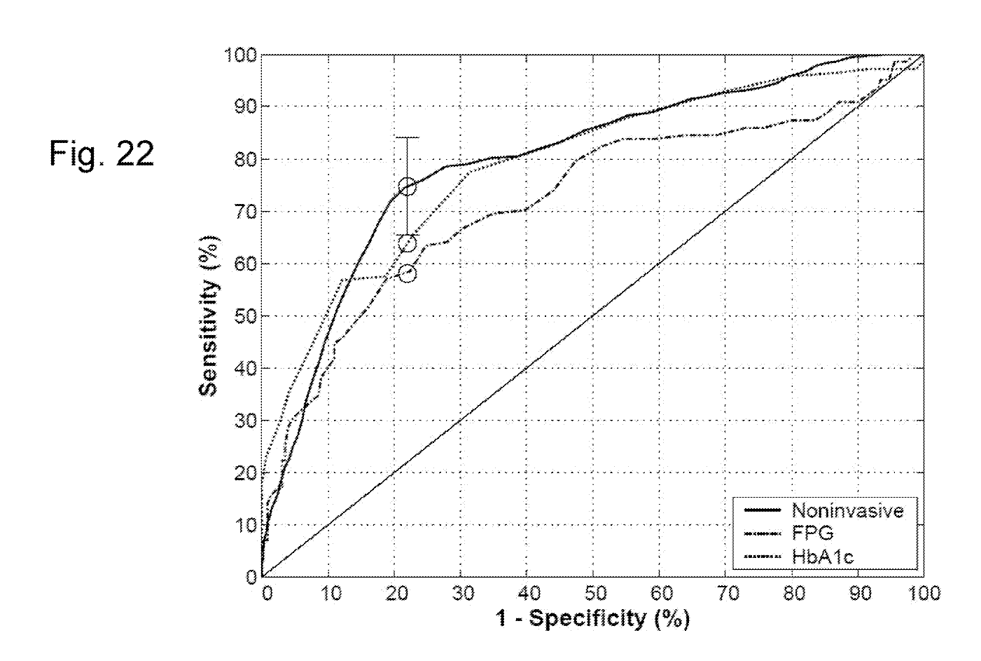

The forearm cradle holds the optical probe and positions a subject's arm properly on the optical probe. The key aspects of the forearm cradle include an ergonomic elbow cup, an armrest and an extendable handgrip. The elbow cup, armrest and handgrip combine to register the forearm properly and comfortably over the optical probe. The handgrip keeps the fingers extended to ensure that forearm is relaxed and reduce muscle tension that might affect the optical measurement. It is also possible to remove the handgrip from the forearm cradle to simplify the instrument without sacrificing overall measurement accuracy. FIG. 20 is a schematic illustration of an example embodiment without a handgrip. In this embodiment, the optical probe is located approximately 3 inches from the elbow to better sample the meaty portion of the volar forearm and provide a good chance of establishing good contact between the volar forearm and the optical probe. This elbow cup/probe geometry allows measurement of a wide range of forearm sizes (2nd percentile female to 98th percentile male). FIG. 20 depicts a commercial embodiment of the instrument and illustrates the volar forearm measurement geometry between the elbow cup 201, optical probe 202 and cradle 203. This version of the commercial embodiment does not have an extendable handgrip, but one can be added if the increased size and complexity is acceptable. In addition, the color and shape of the forearm cradle in the immediate vicinity of the optical probe can be important to attenuate transmission of room lights or other unwanted ambient light through the subject's arm into the detection portion of the optical probe. The color of the forearm cradle in the immediate vicinity of the optical probe can be blue, purple, dark gray or black to attenuate ambient light transmission through a subject's skin and into the optical probe. The forearm cradle can have a concave shape to conform to the curvature of the forearm to partially block ambient light from getting into and under the forearm in a manner that is detectable by the optical probe. The example embodiment also comprises a patient interface 204 and an operator console 205, which comprises a display 206 and a keypad 207.

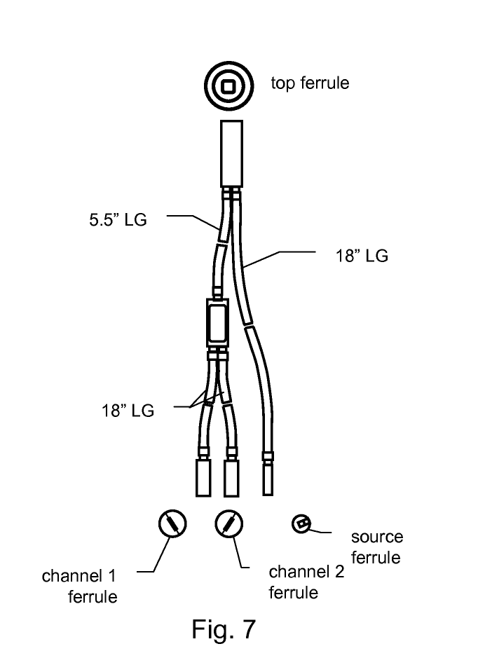

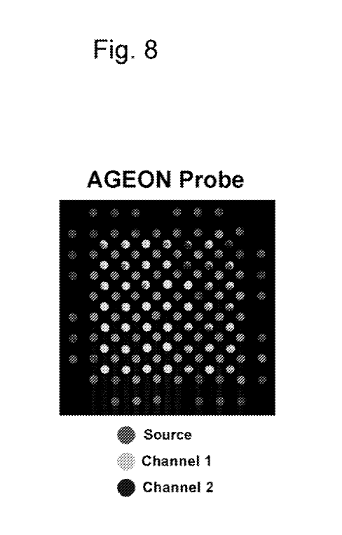

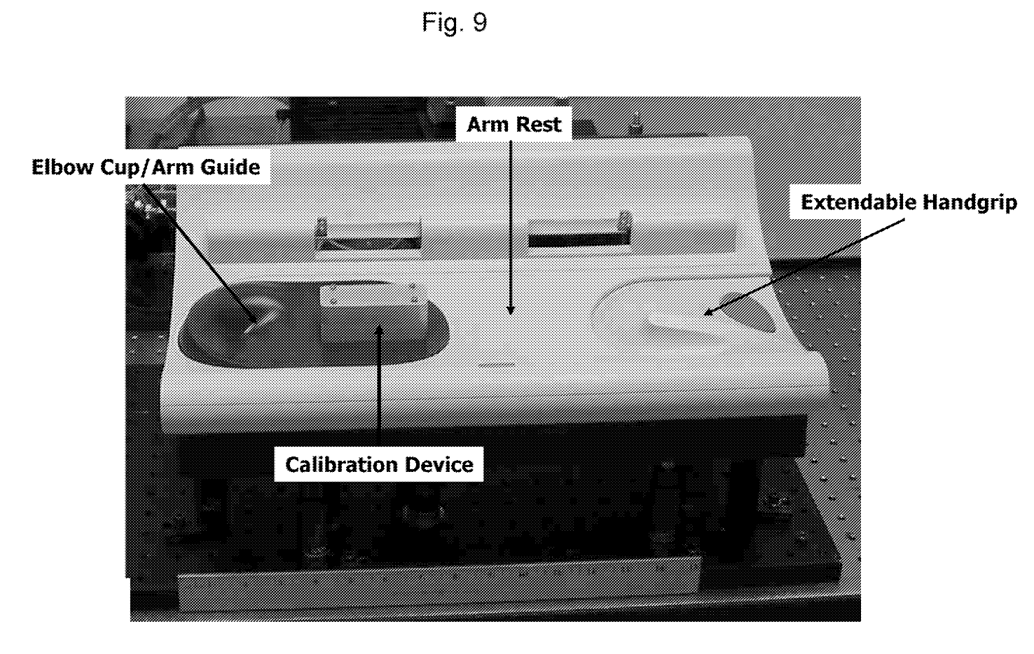

The optical probe is a novel, two detection channel device that uses uniform spacing between the source and receiver fibers to reject surface/shallow depth reflections and target light that reflects or is emitted primarily from the dermal layer of the tissue. FIG. 7 is a schematic drawing of an example embodiment of an optical probe. The input ferrule of the probe holds fiber optics in a square pattern to match the shape of the square light guide in the light source. The light is conducted to the probe head where it illuminates the tissue of an individual. FIG. 8 shows arrangement of the source and detection channels at the probe head. The source fibers are separated from the detection fibers by a minimum of 80 microns (edge to edge) in order to reject light reflected from the tissue surface. Reflected and emitted light from the beneath the skin surface is collected by the detection channels and conducted to separate inputs of a spectrograph. The two detection channels have different but consistent spacing from the source fibers in order to interrogate different depths to the tissue and provide additional spectral information used to detect disease in or assess the health of an individual. The output ferrule of each detection channel arranges the individual fibers in to a long and narrow geometry to match the input slit height and width of the spectrograph. Other shapes are possible and will be driven by the imaging requirements of the spectrograph and the size of the CCD camera used for detection.

It is also possible to run the optical probe in reverse. What were the illumination fibers can become the detection fibers and the two channels of detection fibers become two channels of illumination fibers. This configuration requires two light sources or an optical configuration that can sequentially illuminate the two fiber bundles. It reduces the optical performance requirements of the spectrograph and allows use of a smaller area CCD camera. It also eliminates the need for a mechanical flip mirror in the spectrograph.

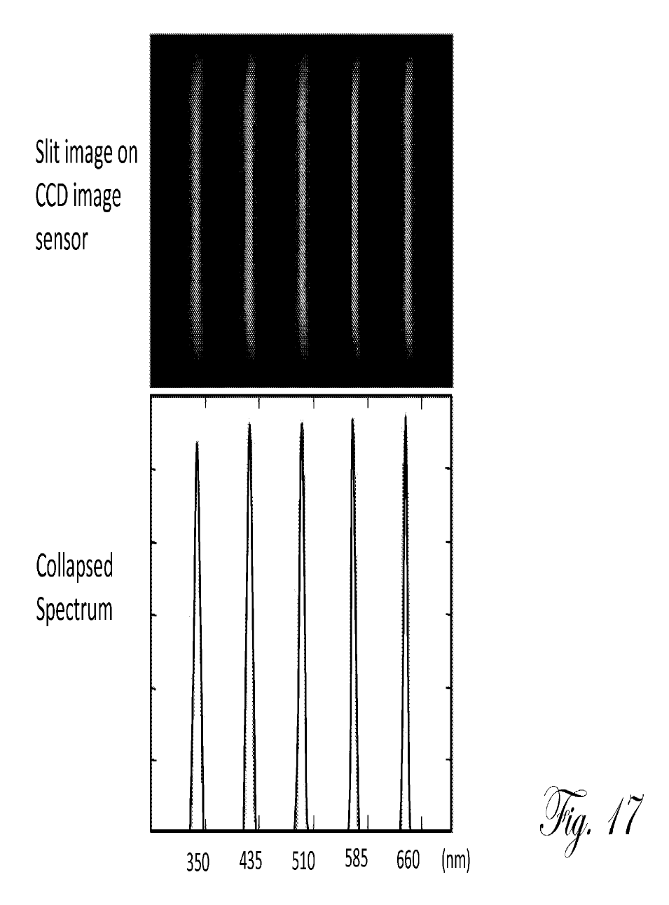

FIG. 14 shows an isometric view of an example embodiment of a trifurcated optical probe having two input illumination channels and one detection channel. The fibers making up each of the illumination channels are bundled together, in this case into a square packed geometry, and match the geometric extent of the light guides of the light source subsystem. Channel 1 utilizes 81 illumination fibers; channel 2 uses 50 illumination fibers. The 50 fibers of the detection channel are bundled together in a 2×25 vertical array, and will form the entrance slit of the spectrograph. In the present example, 200/220/240 micron core/cladding/buffer silica-silica fibers with a 0.22 numerical aperture are used.

The illumination and detection fibers are assembled together at a common plane at the tissue interface. FIG. 15 depicts the relative spatial locations between illumination and detection fibers, where the average center-to-center fiber spacing, (a), from the channel 1 illumination fibers to detection fibers is 0.350 mm, and where the average center-to-center fiber spacing, (b), from the channel 2 illumination fibers to detection fibers is 0.500 mm. The overall extent of fiber pattern is roughly 4.7×4.7 mm. It should be noted that other geometries may be used, having greater or fewer illumination and/or detection fibers, and having a different spatial geometry at the tissue interface.

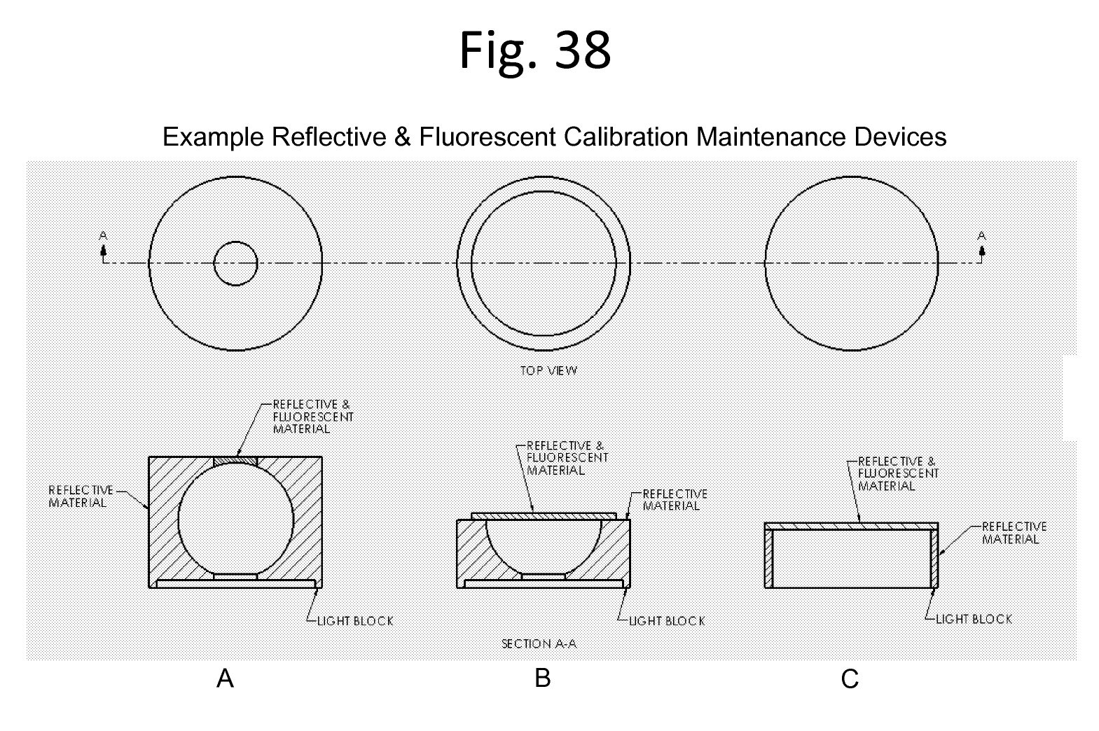

The calibration device provides a reflectance standard (diffuse or otherwise) that is periodically placed on the optical probe to allow measurement of the overall instrument line shape. The measurement of the instrument line shape is important for calibration maintenance and can be used to compensate for changes/drifts in the instrument line shape due to environmental changes (e.g. temperature, pressure, humidity), component aging (e.g. LEDs, optical probe surface, CCD responsivity, etc.) or changes in optical alignment of the system. Calibration device measurements can also be used to detect if the instrument line shape has been distorted to the point that tissue measurements made with the system would be inaccurate. Examples of appropriate calibration devices include a mirror, a spectralon puck, a hollow integrating sphere made of spectralon, a hollow integrating sphere made of roughened aluminum or an integrating sphere made of solid glass (coated or uncoated). Other geometries besides spherical are also effective for providing an integrated reflectance signal to the detection channel(s) of the optical probe. The common characteristic of all these calibration device examples is that they provide a reflectance signal that is within an order of magnitude of the tissue reflectance signal for a given LED and optical probe channel and that reflectance signal is sensed by the detection portions of the optical probe. In addition, the calibration device can interface with the optical probe in a manner that blocks ambient light (e.g. overhead fluorescent lights) from detection by the optical probe and subsequent contamination of the spectral measurements made with the calibration device. FIG. 38(A,B,C) are schematic illustrations of example calibration maintenance devices suitable for use with some embodiments of the present invention. In some embodiments like those shown in FIG. 38, the calibration device has a skirt that contacts or protrudes below the surface of the optical probe to block ambient light.

Alternately, the calibration device can combine reflectance and fluorescence standards (diffuse or otherwise) into one assembly that is periodically placed on the optical probe to allow measurement of the overall instrument line shape and detect if the instrument is out of calibration. The simultaneous measurement of LED reflectance and the stimulated fluorescence adds extra information for determining if the instrument is in calibration. For example, the ratio of the measured excitation light to the measured fluorescent light can be checked for consistency. In another example, shape-based outlier metrics like spectral F ratio and/or Mahalanobis distance can be calculated for both the excitation and fluorescence light to detect out of calibration conditions. Examples of a calibration device that is both reflective and fluorescent are shown in FIG. 38. A suitable fluorescent material such as USFS-200 or USFS-461 (LabSphere, Inc., USA) can be incorporated into the calibration standard in a manner that allows illumination by the optical probe and collection of both the reflected excitation light as well as the emitted fluorescence. The fluorescent material can be spectralon (LabSphere, Inc, USA) doped with fluorophores that fluoresce in the spectral region of interest for this application, an optionally doped with carbon black to reduce the reflectivity (1% to 98% reflectivity) of the spectralon surface to mimic the amount of light returned from tissue. Preferentially, the fluorescent material is stable over time and is not prone to photo-bleaching. In FIG. 38A, the fluorescent material is a plug that can be inserted into a calibration device that has an integrating sphere geometry, providing superior diffuse reflection and even detection by the optical probe. In an alternate embodiment shown in FIG. 38B, the fluorescent material comprises the optically active top of the calibration device combined with a diffusely reflective hemisphere. As a further example shown in FIG. 38C, the fluorescent material can be used to provide both the reflectance and fluorescence. Other embodiments that provide a combination of excitation light reflectance and resulting fluorescence emission are possible.

The calibration device can be used to measure the instrument line shape for each LED and the neon lamp of the illumination subsystem for each input channel of the optical probe. The measured neon lamp line shape is especially useful for detecting and correcting for alignment changes that have shifted or otherwise distorted the x-axis calibration of the instrument because the wavelengths of the emission lines of the neon gas are well known and do not vary significantly with temperature. The measurement of each LED for each optical probe channel can be used to determine if the instrument line shape is within the limits of distortion permitted for accurate tissue measurements and, optionally, can be used to remove this line shape distortion from the measured tissue spectra to maintain calibration accuracy. Line shape removal can be accomplished by simple subtraction or ratios, with optional normalization for exposure time and dark noise.

The spectrograph disperses the light from the detection channels into a range of wavelengths. In the example of FIG. 1, the spectrograph has a front and side input that utilizes a flipper mirror and shutter to select which input to use. The input selection and shutter control is done by computer. The spectrograph uses a grating (i.e. a concave, holographic grating or a traditional flat grating) with blaze and number of grooves per inch optimized for the spectral resolution and spectral region needed for the noninvasive detection of disease. In the current example, a resolution of 5 nm is sufficient, though higher resolutions work just fine and resolution as coarse as 2520 nm will also work. The dispersed light is imaged onto a camera (CCD or otherwise) for measurement.

FIG. 16 depicts an example embodiment of the spectrograph. It is composed of a single concave diffraction grating having two conjugate planes defining entrance slit and image locations. The concave diffraction grating collects light from the entrance slit, disperses it into its spectral components, and reimages the dispersed spectrum at an image plane. The grating can be produced via interferometric (often call holographic) or ruled means, and be of classical or aberration corrected varieties.

The detection fibers of the optical probe are bundled into a 2×25 array and can define the geometry of the entrance slit. The fiber array is positioned such that the width of the slit defined by the 2 detection fibers in the array lies in the tangential plane (in the plane of the page), and the height of the slit defined by the 25 fibers of the array lie in the sagittal plane (out of the plane of the page).

In addition to allowing the array of detection fibers to define the entrance slit, an auxiliary aperture, such as two knife edges or an opaque member with appropriate sized opening, can be used. In this configuration, the fiber array would be brought into close proximity with the aperture so as to allow efficient transmission of light through the aperture. The size of the aperture can be set to define the spectrometer resolution.

The detection fiber array can also be coupled to the entrance slit of the spectrometer with a light guide. An appropriately sized light guide matching the geometric extend of the 2×25 detection fiber array, e.g. 0.5×6 mm, and having a length of at least 20 mm can be used, having an input side coupled to the fiber array and an output side that can either define the entrance slit of the spectrometer or coupled to an aperture as described previously. The light guide can take the form of a solid structure, such as a fused silica plate, or of a hollow structure with reflective walls. The light guide can be particularly useful when considering calibration transfer from one instrument to another because it reduces the tolerance and alignment requirements on the detection fiber array by providing a uniform input to the spectrograph slit.

In the current example the diffraction grating is capable of dispersing light from 360 to 660 nm over a linear distance of 6.9 mm, matching the dimension of a CCD image sensor. FIG. 17 shows an example of an image formed onto the CCD image sensor with multiple wavelengths of 360, 435, 510, 585, and 660 nm, and the corresponding spectrum produced by vertically binning the pixels of the CCD shown below. Gratings with other groove densities can be used depending on the desired spectral range and size of the image sensor.

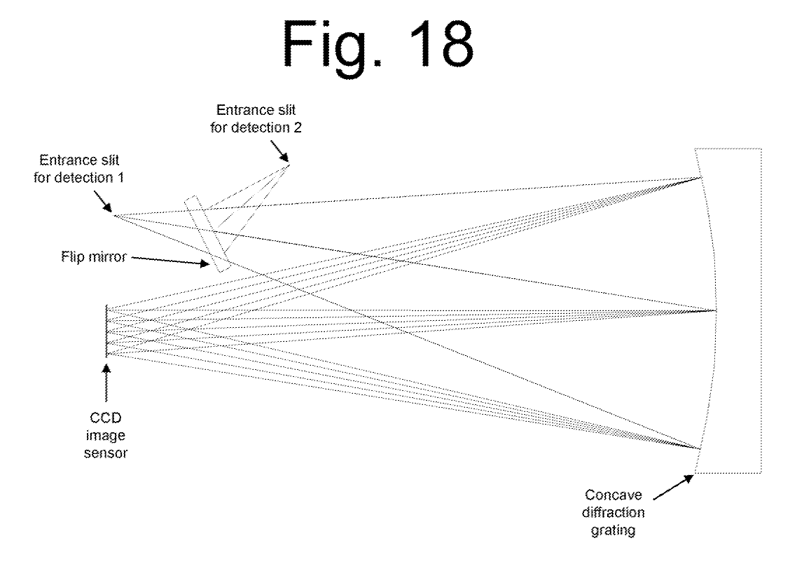

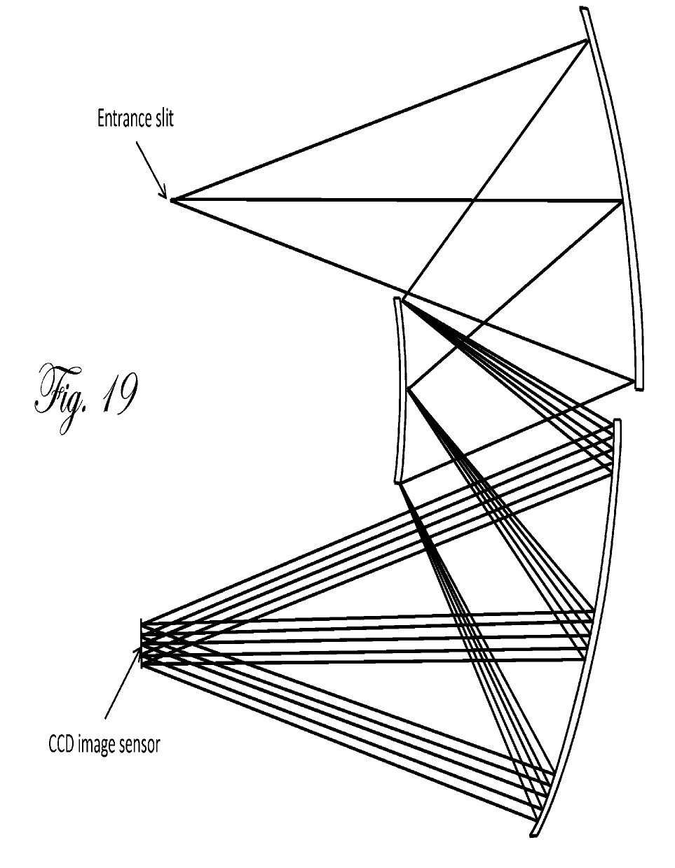

A previously disclosed optical probe described having two detection channels. While the aforementioned spectrometer identifies a single entrance slit to interface with a single detection channel of an optical probe, it is possible to design the spectrometer to accept multiple inputs. FIG. 18 depicts another embodiment in which a flip mirror is used to change between one of two entrance slits. The location of each entrance slit is chosen so that they have a common conjugate at the image plane. In this manner, one can chose between either of the two inputs to form a spectral image of the corresponding detection channel.

One skilled in the art will realize that other mounts, gratings, and layout designs may be used with similar intent. FIG. 19 shows just one example, that of an Offner spectrograph having primary and tertiary concave mirrors, and a secondary convex diffraction grating. The Offner spectrometer is known to produce extremely good image quality as there are sufficient variables in the design to correct for image aberrations, and therefore has the potential of achieving high spectral and spatial resolution. Other examples of suitable spectrograph designs may include, but are not necessarily limited to, Czerny-Turner, Littrow, transmission gratings, and dispersive prisms.

While there are many spectrograph designs to choose from, certain configurations can be more desirable than others depending on the desired characteristics of the system Those requirements can include items such as cost, size, performance, and etendue (or throughput). In one example, the system is desired to have low cost and small size while maintaining high performance and throughput, and a spectrometer based on a fast (e.g. F/2) concave holographic grating and front-illuminated CCD image sensor, such as the embodiment depicted in FIG. 16, has the potential to meet these requirements. This configuration is well known and there are many commercially available gratings and mounts of this type to choose from. In this configuration, the entrance slit and CCD are located in a common plane, creating bilateral symmetry about a plane in the page and bisecting the system into a top half and bottom half (i.e. through the center of the entrance slit and CCD), and is often referred to as an in-plane grating design. In spite of the appeal in its ability to meet several design requirements, this typical in-plane spectrograph design can suffer from stray light that can dramatically impact overall system performance, as described below.

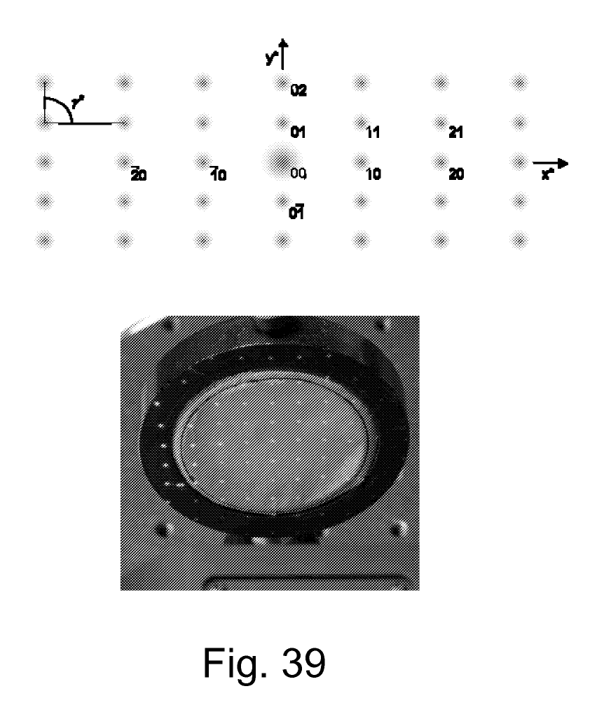

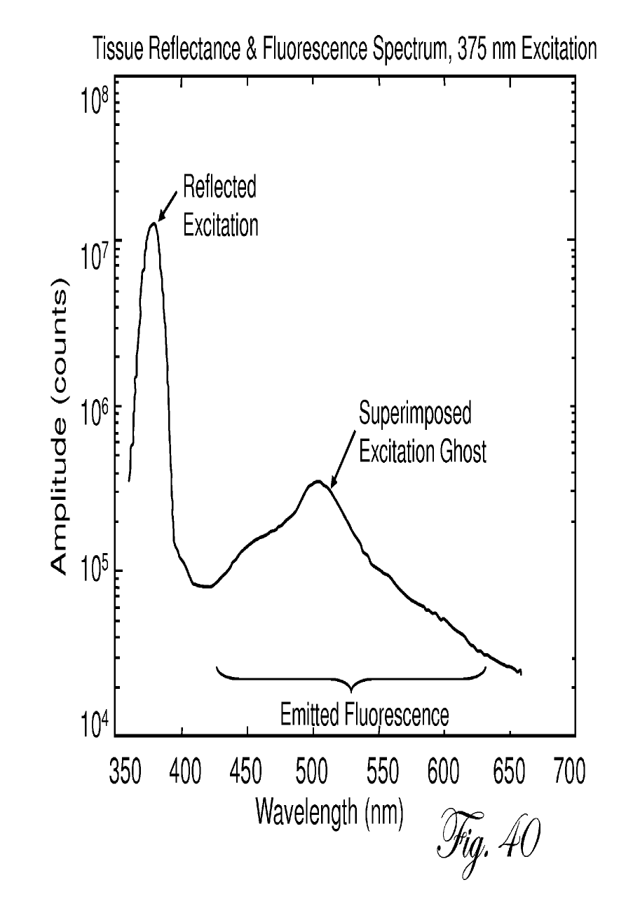

Due to the high refractive index of the silicon substrate, not all of the light striking the CCD image sensor is detected and converted to an electronic signal. A significant portion of the light is reflected and diffracted off the CCD, and the two-dimensional structure of the CCD pixel array creates a two-dimensional diffraction pattern, as shown in FIG. 39. This diffracted light is returned back into the spectrograph and can result in a stray light signal corrupting the desired measurement signal (see, e.g., Richard W. Bormett and Sanford A. Asher, “2-D Light Diffraction from CCD and Intensified Reticon Multichannel Detectors Causes Spectrometer Stray Light Problems”, Applied Spectroscopy, Volume 48, Number 1, January 1994, pp. 1-6(6)1, incorporated herein by reference. In a bilaterally symmetric, in-plane spectrometer configuration such as that depicted in FIG. 16, this stray light can actually result in a ghost signal in the form of a secondary slit image on the CCD. For example, light can take the following path through the spectrometer: light emerges from entrance slit and propagates to the grating, the −1 diffracted order from the grating is imaged onto the CCD generating the desired signal, a portion of this light is diffracted off the CCD (such as orders −4<m<4) back toward the grating in a two-dimension array, the grating collects and re-diffracts this light and the m=−3 grating order is reimaged back onto the CCD, but spatially separated from the primary signal. This doubly-diffracted ghost signal, while lower in intensity than the primary signal, can be undesirable and can detract from overall system performance because its spectral location can overlap the detected fluorescence and can be of similar amplitude, artificially inflating the apparent size and shape of the fluorescence, as shown in FIG. 40. This can be particularly detrimental if the reflectance ghost is not related to the detection of tissue state or disease state because it interferes with the fluorescence measurement.

The bilateral symmetry of the in-plane grating design discussed previously is a cause of the ghost signal generation. This symmetric geometry allows for stray light to propagate back and forth between the CCD and grating. In order to reduce or eliminate the ghost signal other design options can be desirable. For example, a back-illuminated CCD image sensor which is tilted away from the grating can be used. The back illuminated CCD can have a smooth surface, eliminating the two-dimensional diffraction pattern that is generated from the pixel array of a front illuminated CCD. Additionally, the light that is specularly reflected off the CCD surface reflects away from the grating when the CCD is appropriately tilted. An anti-reflection coating can be applied to the CCD silicon surface to reduce the magnitude of the reflected light. In this manner, an in-plane grating design can be used and achieve a reduced or eliminated ghost signal. However, back illuminated CCD's can be significantly more expensive, potentially prohibitive when cost is an important factor.

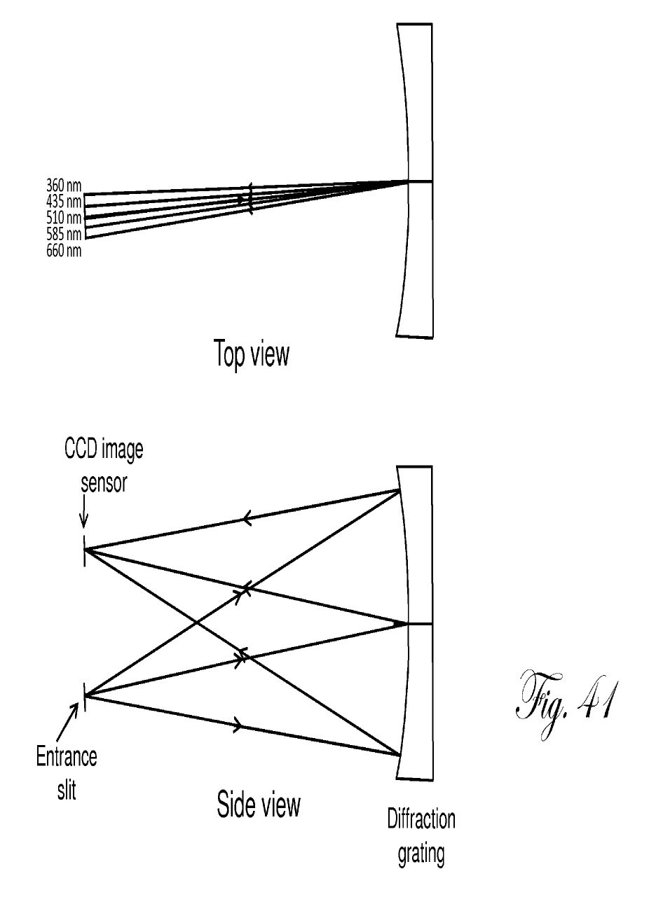

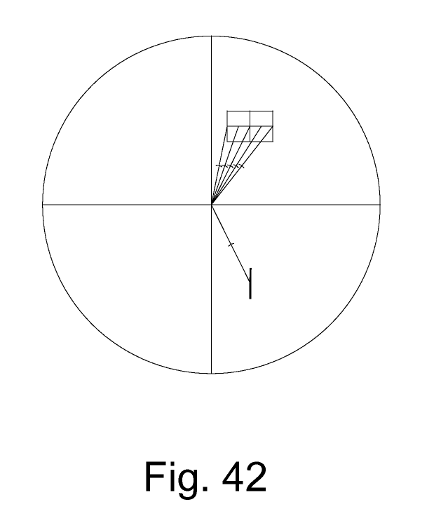

As another example, an alternate spectrograph design that breaks the symmetry of the in-plane design can be used. An example of one such solution is an out-of-plane Littrow mount design as shown in FIG. 41. In the Littrow configuration, the incoming and diffracted beams are coincident or nearly coincident (i.e. the diffracted beam comes back on the input beam), as depicted in the top view of FIG. 41. Rotating to the side view of FIG. 41, the entrance slit and image planes have been spatially separated in order to fit an image sensor to enable spectral collection. FIG. 42 shows an end-on view looking toward the concave surface of the grating. Note that the bilateral symmetry of the in-plane design has been broken, and the entrance slit and image plane are located above and below one another. With an in-plane design, for example, the entrance slit can be located on the negative x-axis while the image plane is located on the positive x-axis. The XZ plane then defined a plane of symmetry. With this Littrow mount, light that is specularly reflected (or zero-order diffraction) off the CCD propagates away from the grating. This can be appreciated by considering the side view of FIG. 41, where the reflected beam off the CCD returns above, but does not strike, the grating. A number of beams from the 2D diffraction pattern off the CCD will still strike the grating and consequently will be diffracted and reimaged. However, that secondary beam does not return to the CCD, but is reimaged back at the entrance slit. In this way, the ghost signal at the CCD has been completely eliminated. Several diffraction grating designs may be used in a Littrow mount configuration, including, but not limited to, ruled and holographic gratings, Rowland circle gratings, and aberration corrected grating designs. The appropriate grating design can depend on desired cost, spectrometer geometry and performance requirements.

The CCD camera subsystem measures the dispersed light from the spectrograph. All wavelengths in the spectral region of interest are measured simultaneously. This provides a multiplex advantage relative to instruments that measure one wavelength at a time and eliminates the need to scan/move the grating or detector. The exposure time of the camera can be varied to account for the intensity of the light being measured. A mechanical and/or electrical shutter can be used to control the exposure time. The computer subsystem instructs the camera as to how long an exposure should be (10's of milliseconds to 10's of seconds) and stores the resulting image for later processing. The camera subsystem can collect multiple images per sample to allow signal averaging, detection of movement or compensation for movement/bad scans. The CCD camera should have good quantum efficiency in the spectral region of interest. In the current example, the CCD camera is responsive to light in the 250 to 1100 nm spectral range.

The computer subsystem controls the operation of the light source, spectrograph and CCD camera. It also collects, stores and processes the images from the camera subsystem to produce an indication of an individual's disease status based on the fluorescence and reflectance spectroscopic measurements performed on the individual using the instrument. As shown in FIG. 20, an LCD display and keyboard and mouse can serve as the operator interface. Alternately, the operator interface can be simplified by combining an LCD display with a touchscreen. The operator interface can be rotated in azimuth and elevation to allow the operator to adjust the position for patient comfort, optimal data entry and instrument control. There can be additional indicators on the instrument to guide the patient during a measurement. In addition, audio output can be used to improve the usability of the instrument for patient and operator.

Compensation for Competitive Signal

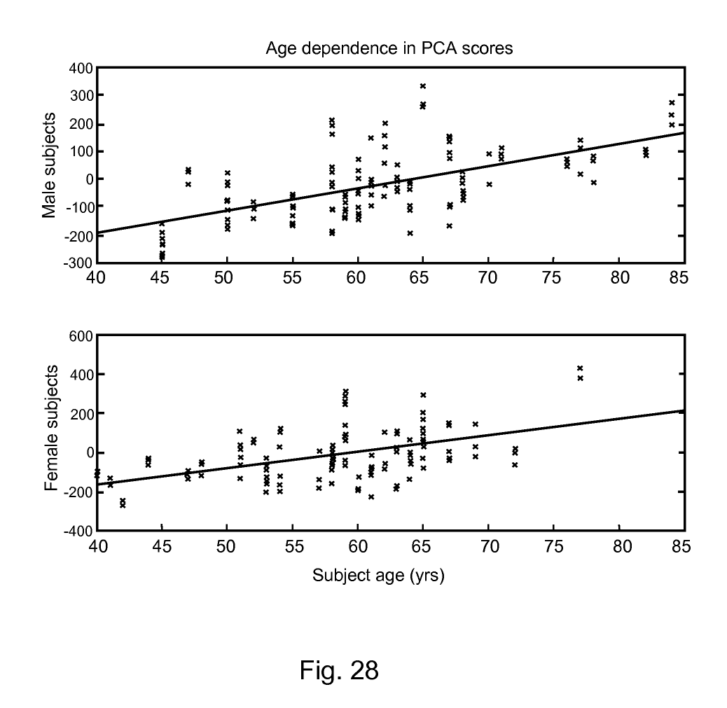

Compensation for competitive signal refers to techniques for removing or mitigating the impact of predictable signal sources that are unrelated to and/or confound measurement of the signal of interest. As compared to multivariate techniques that attempt to “model through” signal variance, this approach characterizes signal behavior that varies with a quantifiable subject parameter and then removes that artifact. One example of such a signal artifact is the age-dependent variation of skin fluorescence. Because of signal overlap between skin fluorescence due to age and similar fluorescence signals related to disease state, uncompensated signals can confuse older subjects without disease with younger subjects with early stage disease (or vice versa). FIG. 28 illustrates the dependence of skin fluorescence with the age of an individual.

Similar competitive effects may be related to other subject parameters (e.g., skin color, skin condition, subject weight or body-mass-index, etc). Numerous techniques exist for modeling and compensation. Typically, a mathematical algorithm is established between signal and the parameter based upon measurements in a controlled set of subjects without disease or health condition. The algorithm can then be applied to new subjects to remove the signal components relating to the parameter. One example relates to compensation for age-dependent skin fluorescence prior to discriminant analysis to detect disease or assess health. In this approach, the spectra from subjects without disease are reduced to eigen-vectors and scores through techniques such as singular-value decomposition. Polynomial fits between scores and subject ages are computed. Scores of subsequent test subject spectra are adjusted by these polynomial fits to remove the non-disease signal component and thus enhance classification and disease detection performance.

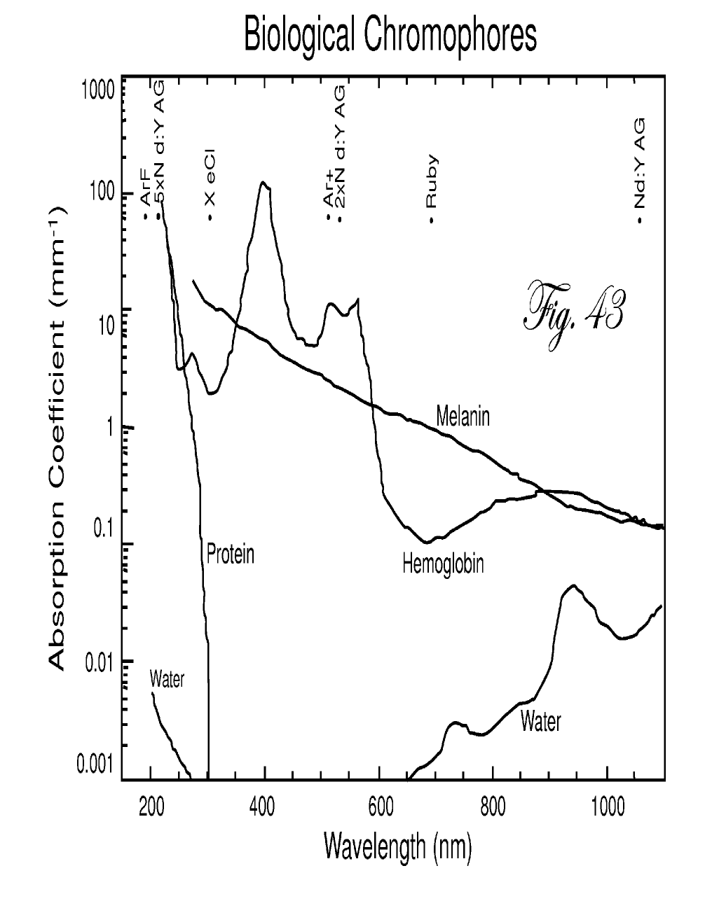

Over the 250 nm to 900 nm spectral region, the dominant absorbers of light in skin are melanin and hemoglobin. FIG. 43 shows the absorption coefficients of melanin, hemoglobin, water and protein (i.e. collagen, elastin) over the 150 nm to 1100 nm spectral region. The amount of melanin, hemoglobin, water and protein contained in skin is subject dependent and must be taken into account when making reflectance and fluorescence measurements. The intrinsic fluorescence correction technique described in U.S. Pat. No. 7,139,598 is an example method of compensating for these subject specific differences. The method can compensate for the static concentrations of melanin, hemoglobin, water and protein in the skin of an individual as well as short term dynamic changes in hemoglobin. In the context of the present description, static is taken to mean the concentration of a given chromophore does not change significantly during the course of a measurement, while a dynamic change is one that occurs during the course of a measurement.

The method can compensate for dynamic changes in the measurement due to hemoglobin variation that follows the heart beat of a subject by taking measurements over a sufficient period of time to average out this variation and by collecting excitation LED skin reflectance simultaneously with LED skin fluorescence. The averaging can be effective for compensating for the time separation between the measurement of the white LED used to characterize skin reflectance in the fluorescence emission spectral region and the measurement of the excitation LED reflectance and emitted fluorescence. The amount of time averaged can be approximately 6 seconds to capture and average between 4 and 12 beats of the heart. In order to achieve this total measurement time, a combination of exposures and pulse width modulation allows the method to be used on a wide variety of subjects whose measured light can vary by three or more orders of magnitude. As an example, if 6 seconds of measurement are desired to reduce signal fluctuations due to the hemoglobin and the beating of the heart, four 1.5 second exposures can be collected in rapid series. If the subject is very fair skinned, there is the potential to saturate the camera during the 1.5 second exposure time, so pulse width modulation can be used to reduce the apparent brightness of the LED and keep the camera from being saturated at the excitation wavelengths. If the subject is dark skinned, the LED can be turned on continuously (no pulse width modulation) and the exposure time extended (e.g. up to N seconds) to achieve the desired signal to noise ratio for the measurement. This is just one example of how programmable pulse width modulation and exposure time can be used to achieve optimal signal-to-noise ratios and maintain measurement precision and accuracy.

The method can compensate for static differences in the amount of light returned by a given subject in a particular measurement by first measuring the light return for each LED or light source using a very short time exposure measurement (e.g. 50 ms hot shot) of the skin. Subsequent exposures for the particular LED can be scaled in time and degree of pulse width modulation based on the initial short time exposure measurement (hot shot) and the well depth (max counts) of the camera (i.e. pulse width modulation duty cycle=(measured counts/max counts)*(hot shot exposure time/desired measurement exposure time)) to achieve a certain signal level on the camera that optimizes the signal-to-noise ratio of the measurement. The measurement can then be normalized to camera counts per second by taking the measured counts and dividing that quantity by the product of the exposure time in seconds and the pulse width modulation duty cycle. As an example, if the pulse width modulation duty cycle is 50% and the exposure time was 1.0 seconds for a 50,000 count measurement for a given pixel of the camera, then the counts per second would be 50,000/(0.5 * 1.0)=100,000 counts/second for that camera pixel.

Combining Classification Techniques

The technique described here improves classification performance by combining classifications based upon different disease thresholds and/or applying a range of classification values rather than simply binary (one or zero) choices. Typical disease state classification models are built by establishing multivariate relationships in a calibration data set between spectra or other signals and a class value. For example, a calibration subject with the disease or condition can be assigned a class value of one while a control subject has a class value of zero. An example of the combined classification methods is to create multiple class vectors based upon different disease stages. Separate discriminant models can then be constructed from the data set and each vector. The resulting multiple probability vectors (one from each separate model) can then be bundled or input to secondary classification models to yield a single disease probability value for each sample. Bundling refers to a technique of combining risk or probability values from multiple sources or models for a single sample. For instance, individual probability values for a sample can be weighted and summed to create a single probability value. An alternative approach to enhance classification performance is to create a multi-value classification vector where class values correspond to disease stages rather than the binary value (one/zero). Discriminant algorithms can be calibrated to compute probability into each non-control class for optimal screening or diagnostic performance.

Sub-Modeling

Sub-modeling is a technique for enhancing classification or quantification model performance. Many data sets contain high signal variance that can be related to specific non-disease sample parameters. For example, optical spectra of human subjects can encompass significant signal amplitude variations and even spectral shape variations due primarily to skin color and morphology. Subdividing the signal space into subspaces defined by subject parameters can enhance disease classification performance. This performance improvement comes since subspace models do not have to contend with the full range of spectral variance in the entire data set.

One approach to sub-modeling is to identify factors that primarily impact signal amplitude and then develop algorithms or multivariate models that sort new, test signals into two or more signal range categories. Further grouping can be performed to gain finer sub-groupings of the data. One example of amplitude sub-modeling is for skin fluorescence where signal amplitude and optical pathlength in the skin is impacted by skin melanin content. Disease classification performance can be enhanced if spectral disease models do not have to contend with the full signal dynamic range. Instead, more accurate models can be calibrated to work specifically on subjects with a particular range of skin color. One technique for skin color categorization is to perform singular-value decomposition (SVD) of the reflectance spectra. Early SVD factors are typically highly correlated to signal amplitude and subject skin color. Thus, sorting scores from early SVD factors can be an effective method for spectrally categorizing spectra into signal amplitude sub-spaces. Test spectra are then categorized by the scores and classified by the corresponding sub-model.

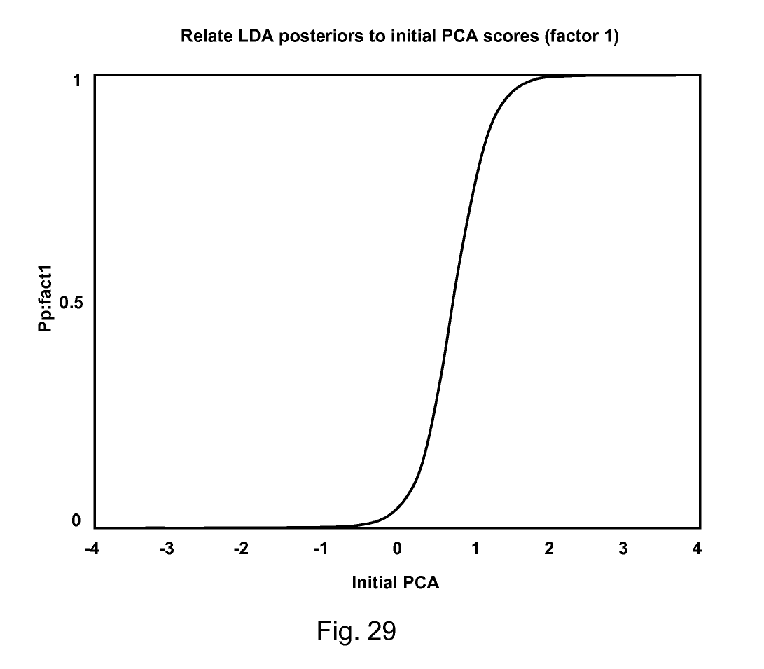

Another sub-modeling method groups spectra by shape differences that correspond to skin color or skin morphology. FIG. 29 illustrates one method of classifying an individual's skin color to help determine which sub-model to employ. Various techniques exist to spectrally sub-divide and then sub-model. Clusters analysis of SVD scores can identify natural groups in the calibration set that are not necessarily related to subject parameters. The cluster model then categorizes subsequent test spectra.

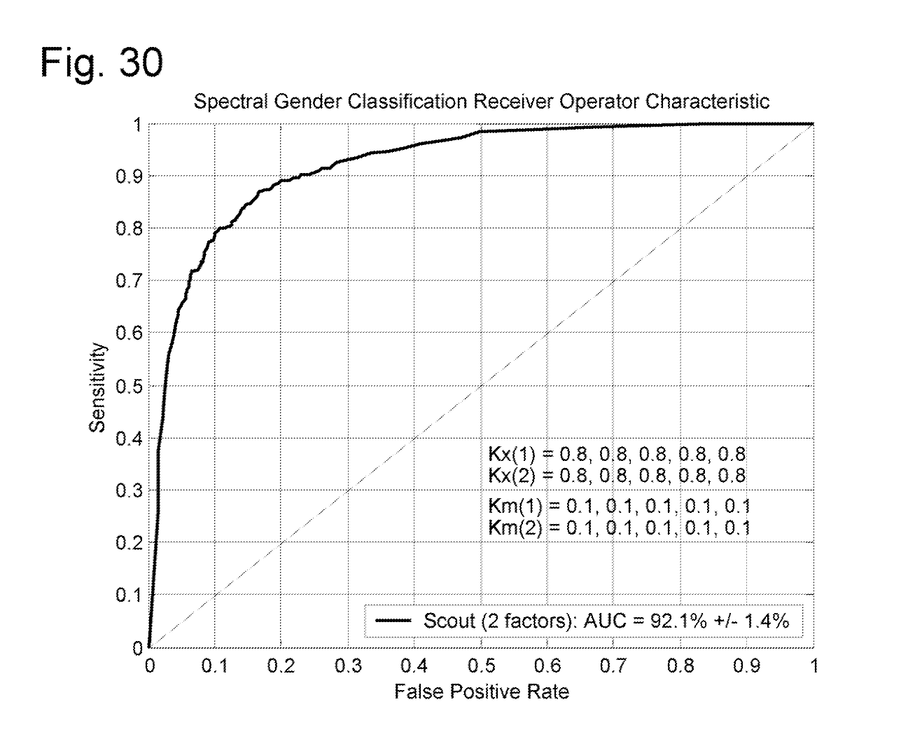

Alternatively, spectral variance can form clusters relating subject parameters such as gender, smoking status, ethnicity, skin condition or other factors like body-mass-index. FIG. 30 shows a receiver operator characteristic of how well genders can be optically separated, with an equal error rate at 85% sensitivity and an area under the curve of 92%. In these instances, multivariate models are calibrated on the subject parameter and subsequent test spectra are spectrally sub-grouped by a skin parameters model and then disease classified by the appropriate disease classification sub-model.

In addition to spectral sub-grouping, categorization prior to sub-modeling can be accomplished by input from the instrument operator or by information provided by the test subject. For example, the operator could qualitatively assess a subject's skin color and manually input this information. Similarly, the subject's gender could be provided by operator input for sub-modeling purposes.

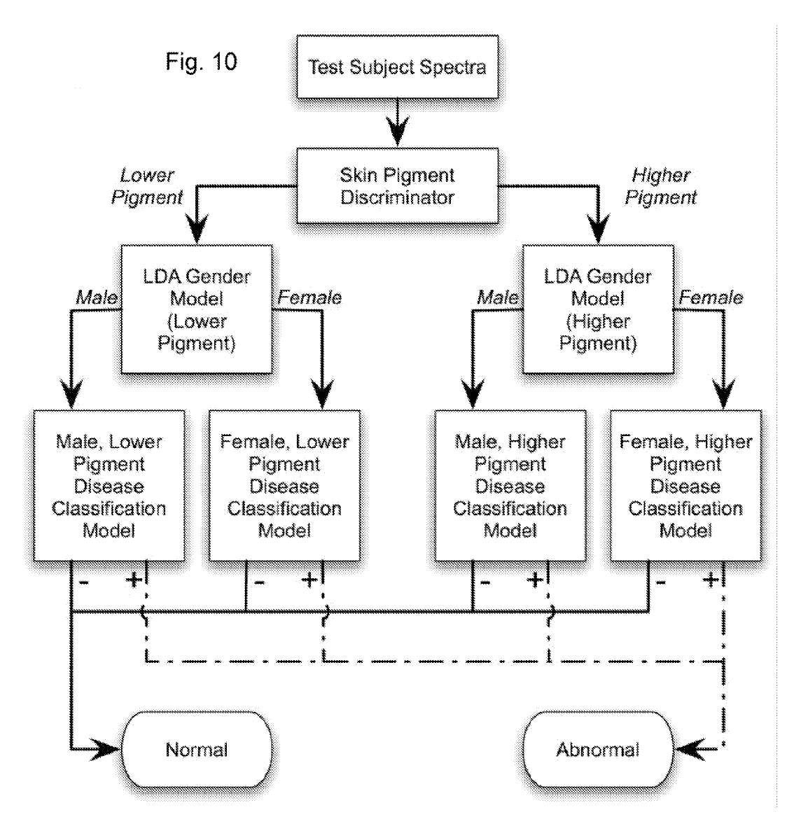

A diagram of a two stage sub-modeling scheme is shown in FIG. 10. In this approach, the test subject's spectra are initially categorized by SVD score (signal amplitude; skin color). Within each of the two skin color ranges, spectra are further sorted by gender discriminant models. The appropriate disease classification sub-model for that sub-group is then applied to assess the subject's disease risk score.

The illustration represents one embodiment but does not restrict the order or diversity of possible sub-modeling options. The example describes an initial amplitude parsing followed by sub-division following gender-based data-clustering. Effective sub-modeling could be obtained by reversing the order of these operations or by performing them in parallel. Sub-groups can also be categorized by techniques or algorithms that combine simultaneous sorting by amplitude, shape or other signal characteristics.

Spectral Bundling

The instrument can produce multiple fluorescence and reflectance spectra that are useful for detecting disease. As an example, a 375 nm LED can be used for both the first and second detection channels of the optical probe, resulting two reflectance spectra that span the 330 nm-650 nm region and two fluorescence emission spectra that span the 415-650 nm region. There are corresponding reflectance and fluorescence emission spectra for the other LED/detection channel combinations. In addition, a white light LED can produce a reflectance spectrum for each detection channel. In an example embodiment there are 22 spectra available for detection of disease.

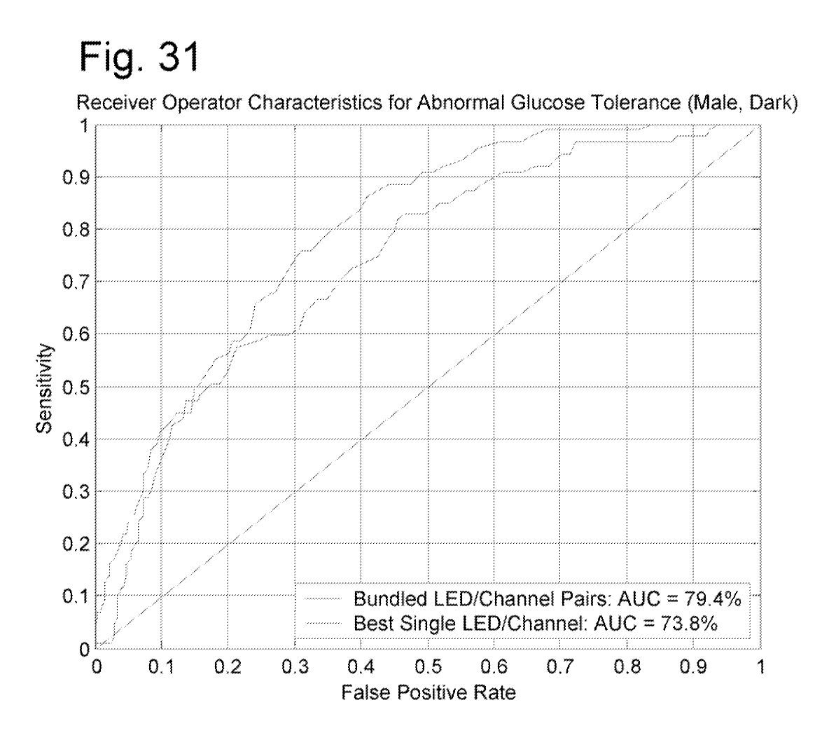

As shown in the receiver operator characteristic of FIG. 31, it is possible to predict disease from a single spectrum for a given LED/detection channel pair, but a single region will not necessarily produce the best overall accuracy. There are several methods of combining the information from each of the LED/detection channel spectral predictions to produce the most accurate overall detection of disease. These techniques include simple prediction bundling, applying a secondary model to the individual LED/detection channel predictions, or combining some or all of the spectra together before performing the analysis.

In a simple bundling technique, disease detection calibrations are developed for each of the relevant LED/detection channel spectra. When a new set of spectra are acquired from an individual, the individual LED/detection channel calibrations are applied to their corresponding spectra and the resulting predictions, PPi (risk scores, posterior probabilities, quantitative disease indicators, etc.), are added together to form the final prediction. The adding of the individual LED/detection channel pairs can be equally (Equation 1) or unequally weighted by a LED/detection channel specific coefficient, ai, (Equation 2) to give the best accuracy.

PP_{bundled}=(Σᵢ₌₁ⁱ⁼ⁿPPᵢ)/n Equation 1

PP_{bundled}=(Σᵢ₌₁ⁱ⁼ⁿaᵢ*PPᵢ)/n Equation 2

The more independent the predictions of the individual LED/detection channel spectra are relative to each other, the more effective the simple bundling technique will be. FIG. 31 is a receiver operator characteristic demonstrating the performance of the simple bundling technique with equal weighting to the individual LED/detection channel predictions.

The secondary modeling technique uses the predictions from the individual LED/detection channel calibrations to form a secondary pseudo spectrum that is input into a calibration model developed on these predictions to form the final prediction. In addition to the LED/detection channel predictions, other variables (scaled appropriately) such as subject age, body mass index, waist-to-hip ratio, etc. can be added to the secondary pseudo spectrum. As an example, if there are 10 distinct LED/detection channel predictions, noted at PP1, PP2 through PP10 and other variables such as subject age, waist to hip ratio (WHR) and body mass index (BMI), a secondary spectrum can comprise the following entries:

Secondary spectrum=[PP1, PP2, PP3, PP4, PP5, PP6, PP7, PP8, PP9, age, WHR, BMI]

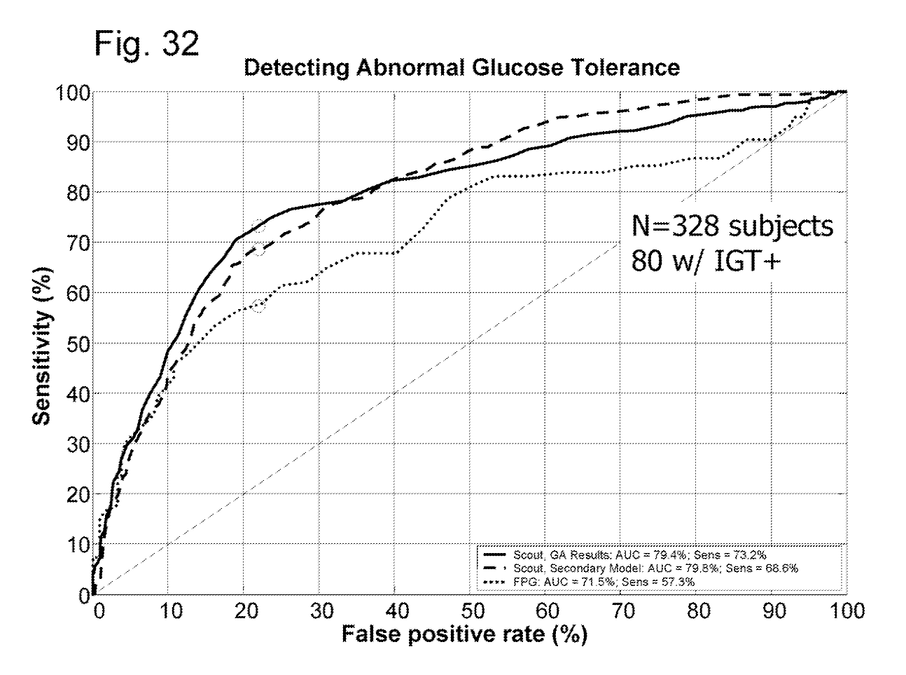

A set of secondary spectra can be created from corresponding fluorescence, reflectance and patient history data collected in a calibration clinical study. Classification techniques such as linear discriminant analysis, quadratic discriminant analysis, logistic regression, neural networks, K nearest neighbors or other like methods are applied to the secondary pseudo spectrum to create the final prediction (risk score) of disease state. FIG. 32 illustrates the performance improvements possible with a secondary model versus simple bundling or a single LED/channel model.

The inclusion of specific LED/detection channel predictions can span a large space (many variations) and it can be difficult to do an exhaustive search of the space to find the best combination of LED/detection channel pairs. In this case, it is possible to use a genetic algorithm to efficiently search the space. See Goldberg, Genetic Algorithms in Search, Optimization and Machine Learning, Addison-Wesley, Copyright 1989 for more details on genetic algorithms. Also, Differential Evolution, ridge regression or other search techniques can be employed to find the optimal combination.

For purposes of the genetic algorithm or differential evolution, the LED/detection channels were mapped to 10 regions (i.e. 375 nm LED/channel 1=region 1; 375 nm LED/channel 2=region 6; 460 nm LED/channel 2=region 10) and the Kx, Km exponents for the intrinsic correction applied to each region we broken into 0.1 increments from 0 to 1.0, yielding 11 possible values for Kx and 11 possible values for Km. The following Matlab function illustrates the encoding of regions and their respective Kx, Km pairs into the chromosome used by the genetic algorithm:

function [region, km, kx]=decode(chromosome)

region(1)=str2num(chromosome(1));

region(2)=str2num(chromosome(2));

region(3)=str2num(chromosome(3));

region(4)=str2num(chromosome(4));

region(5)=str2num(chromosome(5));

region(6)=str2num(chromosome(6));

region(7)=str2num(chromosome(7));

region(8)=str2num(chromosome(8));

region(9)=str2num(chromosome(9));

region(10)=str2num(chromosome(10));

km(1)=min([bin2dec(chromosome(11:14))10])+1;

km(2)=min([bin2dec(chromosome(15:18))10])+1;

km(3)=min([bin2dec(chromosome(19:22))10])+1;

km(4)=min([bin2dec(chromosome(23:26))10])+1;

km(5)=min([bin2dec(chromosome(27:30))10])+1;

km(6)=min([bin2dec(chromosome(31:34))10])+1;

km(7)=min([bin2dec(chromosome(35:38))10])+1;

km(8)=min([bin2dec(chromosome(39:42))10])+1;

km(9)=min([bin2dec(chromosome(43:46))10])+1;

km(10)=min([bin2dec(chromosome(47:50))10])+1;

kx(1)=min([bin2dec(chromosome(51:54))10])+1;

kx(2)=min([bin2dec(chromosome(55:58))10])+1;

kx(3)=min([bin2dec(chromosome(59:62))10])+1;

kx(4)=min([bin2dec(chromosome(63:66))10])+1;

kx(5)=min([bin2dec(chromosome(67:70))10])+1;

kx(6)=min([bin2dec(chromosome(71:74))10])+1;

kx(7)=min([bin2dec(chromosome(75:78))10])+1;

kx(8)=min([bin2dec(chromosome(79:82))10])+1;

kx(9)=min([bin2dec(chromosome(83:86))10])+1;

kx(10)=min([bin2dec(chromosome(87:90))10])+1;

In the example implementation of the genetic algorithm, a mutation rate of 2% and a cross-over rate of 50% were used. Other mutation and cross-over rates are acceptable and can be arrived at either empirically or by expert knowledge. Higher mutation rates allow the algorithm to get unstuck from local maxima at the price of stability.

The population consisted of 2000 individuals and 1000 generations of the genetic algorithm were produced to search the region/Kx/Km space for the optimal combination of regions/Kx/Km. In this particular example the fitness of a given individual was assessed by unweighted bundling of selected region/Kx/Km posterior probabilities (generated previously and stored in a data file which is read in by the genetic algorithm routine for each region and Kx/Km pair per region using methods described in U.S. Pat. No. 7,139,598, “Determination of a measure of a glycation end-product or disease state using tissue fluorescence”, incorporated herein by reference) to produce a single set of posterior probabilities and then calculating a receiver operator characteristic for those posterior probabilities against known disease status. The fitness of a given chromosome/individual was evaluated by calculating classification sensitivity at a 20% false positive rate from the receiver operator characteristic.

The sensitivity at a 20% false positive rate is but one example of an appropriate fitness metric for the genetic algorithm. Other examples would be fitness functions based on total area under the receiver operator characteristic, sensitivity at 10% false positive rate, sensitivity at 30% false positive rate, a weighting of sensitivities at 10, 20 and 30% false positive rates, sensitivity at a given false positive rate plus a penalty for % of outlier spectra, etc. The following Matlab functions are an example implementation of the genetic algorithm:

************************************************************
******
function [ X, F, x, f ] = genetic(chromosomeLength, populationSize, N,
mutationProbability, crossoverProbability)
% --------------------------------------------------------------------------- %
% INPUTS:

| | | |
|---|---|---|
| % | chromosomeLength | (1x1 int) - Number of genes per |

chromosome.

| | | |
|---|---|---|
| % | populationSize | (1x1 int) - Number of chromosomes. |

| | | |
|---|---|---|
| % | N | (1x1 int) - Number of generations. |

| % | mutationProbability (1x1 int) - Gene mutation probability |
|---|---|
| | (optional). |
| % | crossoverProbability (1x1 int) - Crossover probability (optional). |

% OUTPUTS:

| | |
|---|---|
| % | X (1xn char) - Best chromosome over all generations. |
| % | F (1x1 int) - Fitness corresponding to X. |
| % | x (nxm char) - Chromosomes in the final generation. |
| % | f (1xn int) - Fitnesses associated with x. |

% COMMENTS:

| % | populationSize is the initial population size and not the size of |
|---|---|
| % | the population used in the evolution phase. The evolution phase |
| % | of this algorithm uses populationSize / 10 chromosomes. It is |
| % | thus required that populationSize be evenly divisible by 10. In |
| % | addition, because chromosomes crossover in pairs, |
| | populationSize must also be evenly divisible by 2. |

% --------------------------------------------------------------------------- %
if ^{~}exist(‘mutationProbability’, ‘var’)

mutationProbability = 0.02;

end
if ^{~}exist(‘crossoverProbability’, ‘var’)

crossoverProbability = 0.50;

end
% Create the initial population of populationSize chromosomes. Gene
values for
% each chromosome in the initial population are assigned randomly.
rand(‘state’, sum(100 * clock));
rand(‘state’)
for i = 1:populationSize

x(i, :) = num2str(rand(1, chromosomeLength) > 0.5, ‘%1d’);

end
% Trim the initial population by a factor of 10 based on fitness. The
resulting
% population, which will contain populationSize / 10 chromosomes, will
be used
% for the rest of this implementation.
f = fitness(x);
[ Y, l ] = sort(f);
nkeep = populationSize / 10;
nstart = populationSize;
nend = populationSize + 1 − nkeep;
keep_ind = [nstart:−1:nend];
x = x(l(keep_ind),:);
f = f(l(keep_ind));
F = 0;
for i = 1:N

x = select(x, f);
x = crossover(x, crossoverProbability);
x = mutate(x, mutationProbability);
f = fitness(x);
if max(f) > F

F = max(f);
l = find(f == F);
X = x(l, :);

end

end

****************************************************
**************

function y = select(x, f)
p = (f − min(f)) / (max(f − min(f)));
n = floor(p * length(f));
n = ceil(n / (sum(n) / length(f)));
l = [ ];
for i = 1:length(n)
\ l = [ l repmat(i, 1, n(i)) ];
end
l = l(randperm(length(l)));
y = x(l(1:length(f)), :);
************************************************************
******
function f = fitness(chromosome)
for i = 1:size(chromosome, 1)

[ region, km, kx ] = decode(chromosome(i, :));
g = gaFitness(getappdata(0, ‘GADATA’), region, km, kx);
f(i) = g.bsens(2);

end
************************************************************
******
function y = crossover(x, crossoverProbability)
if ^{~}exist(‘crossoverProbability’, ‘var’)

crossoverProbability = 1.0;

end
x = x(randperm(size(x, 1)), :);
y = x;
for i = 1:size(x, 1) / 2

if (rand <= crossoverProbability)

l = floor(rand * size(x, 2)) + 1;
y((2 * i − 1), 1:l) = x((2 * i − 0), 1:l);
y((2 * i − 0), 1:l) = x((2 * i − 1), 1:l);

end

end
************************************************************
******
function y = mutate(x, mutationProbability)
if ^{~}exist(‘mutationProbability’, ‘var’)

mutationProbability = 0.02;

end
y = x;
for i = 1:size(x, 1)

l = find(rand(1, size(x, 2)) <= mutationProbability);
for j = 1:length(l)

if y(i, l(j)) == ‘0’

y(i, l(j)) = ‘1’;

else

y(i, l(j)) = ‘0’;

end

end

end
▪▪▪▪▪▪▪▪▪▪▪▪▪▪▪▪▪▪▪▪▪▪▪▪▪▪▪▪▪▪▪▪▪▪▪▪▪▪▪
▪▪▪▪▪▪▪▪▪▪▪▪▪▪▪▪▪▪▪▪▪▪▪▪▪▪▪▪▪▪▪▪▪▪

FIG. 32 illustrates the performance improvements possible with a genetic algorithm to search the Kx, Km space for each LED/channel pair and selecting regions to bundle.

Another method mentioned above involves taking the spectra from some or all of the LED/detection channel pairs and combining them before generating a calibration model to predict disease. Methods of combination include concatenating the spectra together, adding the spectra together, subtracting the spectra from each other, dividing the spectra by each or adding the log 10 of the spectra to each other. The combined spectra are then fed to a classifier or quantitative model to product the ultimate indication of disease state.

Data Regularization

Before applying any classification technique on a data set, various regularization approaches can be employed, as preprocessing steps, to a derived vector space representation of the spectral data in order to augment signal relative to noise. This normally entails removing or diminishing representative/principal directional components of the data based on their respective variances in the assumption that disease class separation is more likely in directions of larger variance, which is not necessarily the case. These directional components can be defined in many ways: via Singular Value Decomposition, Partial Least Squares, QR factorization, and so on. As a better way to separate signal from noise, one can instead use other information from the data itself or other related data which is germane to disease class separation. One metric is the Fisher distance or similar measure,

$${\{ d ≡ \frac{\vert {u}^{+} - {u}^{-} \vert}{{s}^{2} ( {u}^{+} ) + {s}^{2} ( {u}^{-} )} \}}_{m} ,$$

where u is a data directional component such as a left singular vector, or factor, from SVD. The metric d reveals the degree to which two labeled groups of points are spatially separated from each other in each component of the primary data set studied, which in our case is the spectral data set. In general, however, one can use information from sources outside the spectral data itself as well, such as separate empirical information concerning the relevance of the data components to the underlying phenomena (e.g., similarity of data components to real spectra), their degree of correlation to the data that drives the labeling scheme itself (such as that used for a threshold criterion of disease class inclusion), and so on.

Thus, for each data component, we can use, e.g., Fisher distance to weigh that component relative to the others or eliminate it altogether. In so doing, data components are treated differently from one another: those which demonstrate greatest separation between disease classes, or otherwise show greatest relevance to disease definition, are treated most favorably, thereby increasing the ability of a subsequently applied classification technique to determine a good boundary between disease and non-disease points in the data space. To each directional SVD component we multiply a severity-tunable filter factor such as

$${F}_{j} = \frac{{d}_{j}}{{d}_{j} + γ}$$

where dj is the Fisher distance, or any metric or other information of interest, for the jth directional component/factor, and γ is a tuning parameter which determines the degree to which the data components are treated differently. A search algorithm can be employed to find γ such that the performance of any given classifier is optimal.

Such a regularization approach can produce notable improvement in the performance of a classifier, as can be seen from the change in the ROC (Receiver Operating Characteristic) curve in Support Vector Regression (SVR), or Kernel Ridge Regression (KRR) based classification for skin fluorescence spectra shown below. See, e.g., The Nature of Statistical Learning Theory, Vladimir N. Vapnik, Springer-Verlag 1998; T. Hastie, R. Tibshirani, and J. H. Friedman, The Elements of Statistical Learning, Springer 2003; Richard O. Duda, Peter E. Hart, and David G. Stork, Pattern Classification (2nd Edition), Wiley-Interscience 2000. The details of the SVR/KRR based approach are examined below.

Regularization Results for SVR Classification

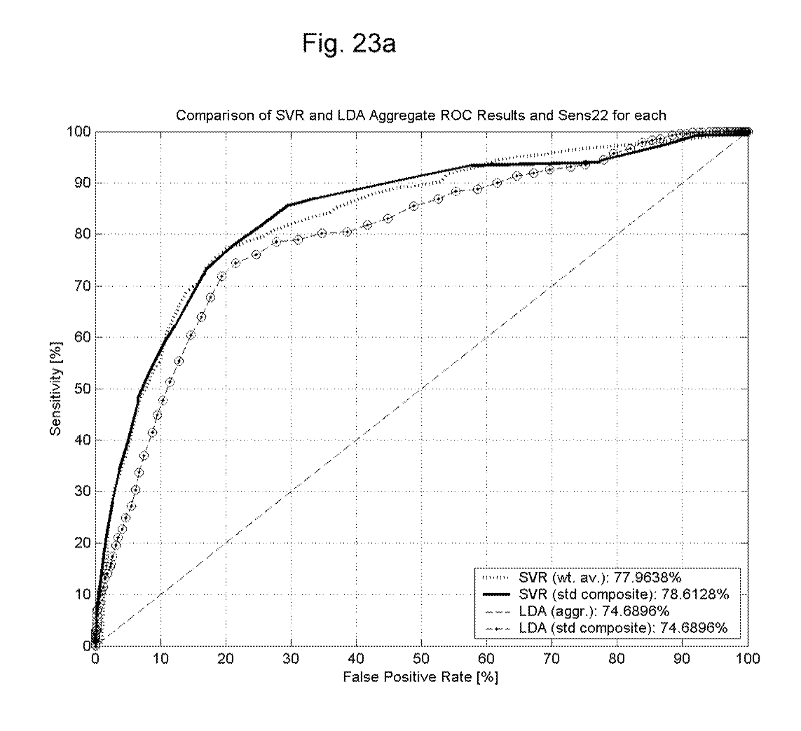

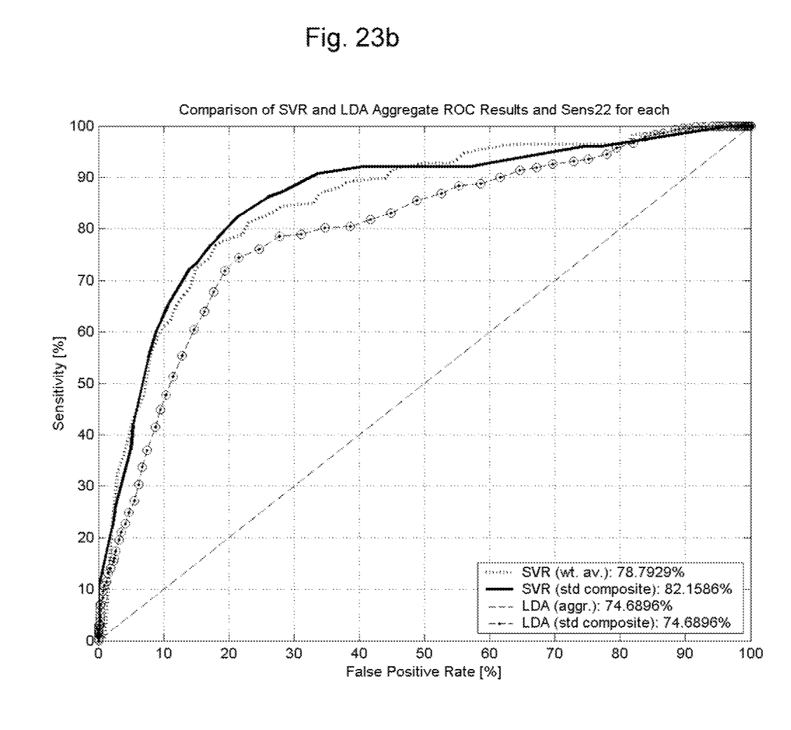

The results of disease detection sensitivity for the two cases of regularization, as defined by Fj above, and no-regularization are shown in FIG. 23-27 for the DE(SVR) wrapper classification technique in the form of ROC curves. The SVR results are based on spectral data which was age-compensated (see Compensation for Competitive Signal) inside a cross validation protocol. All other preprocessing in SVR, including regularization, was also done to each fold of a cross validation protocol for model stability and robustness. Previous results of regularized Linear Discriminant Analysis [GA(LDA)] are included as a reference. Regularization for GA(LDA) involved removal of SVD components ranked low in Fisher distance, as opposed to being weighted by Fj. The overall classification model was produced by the combined sub-model approach outlined in the Submodeling section.

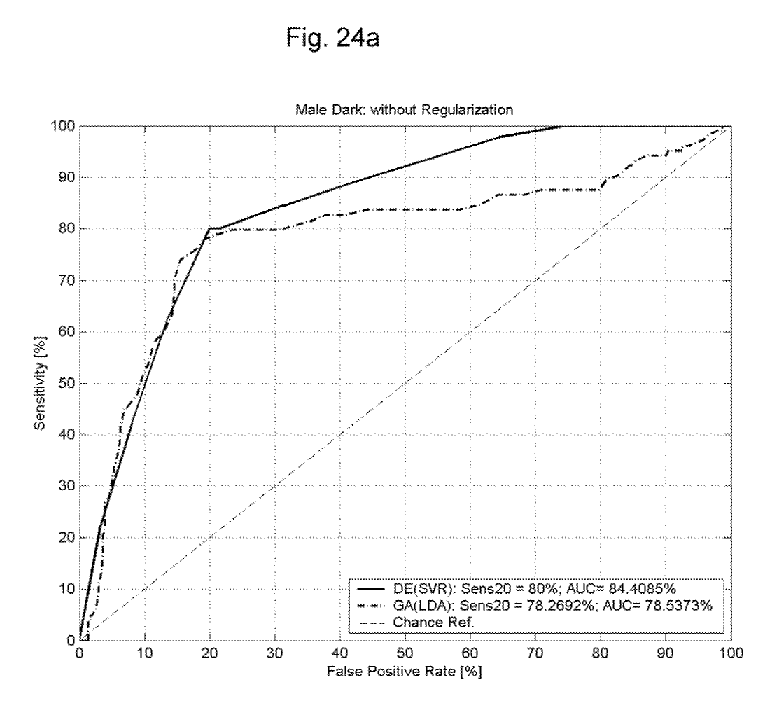

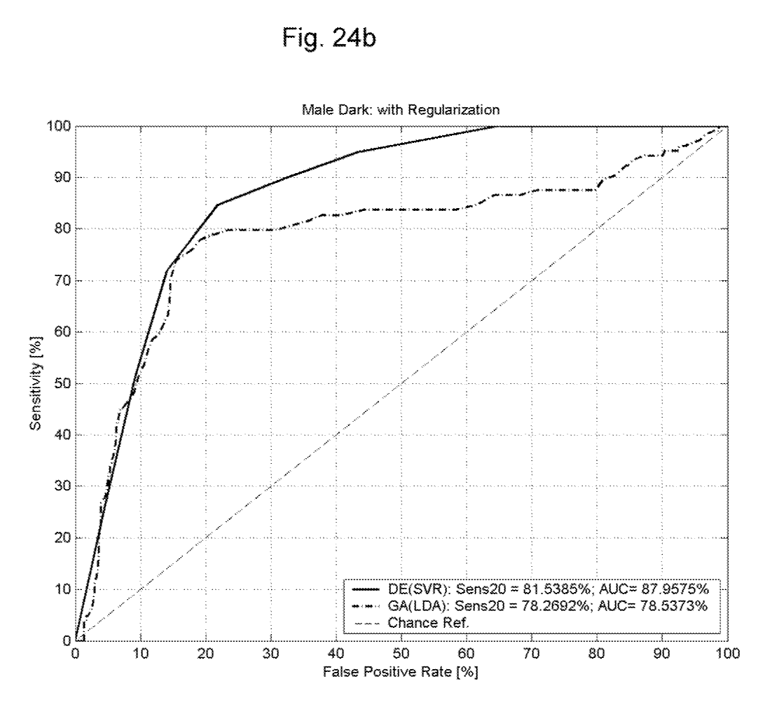

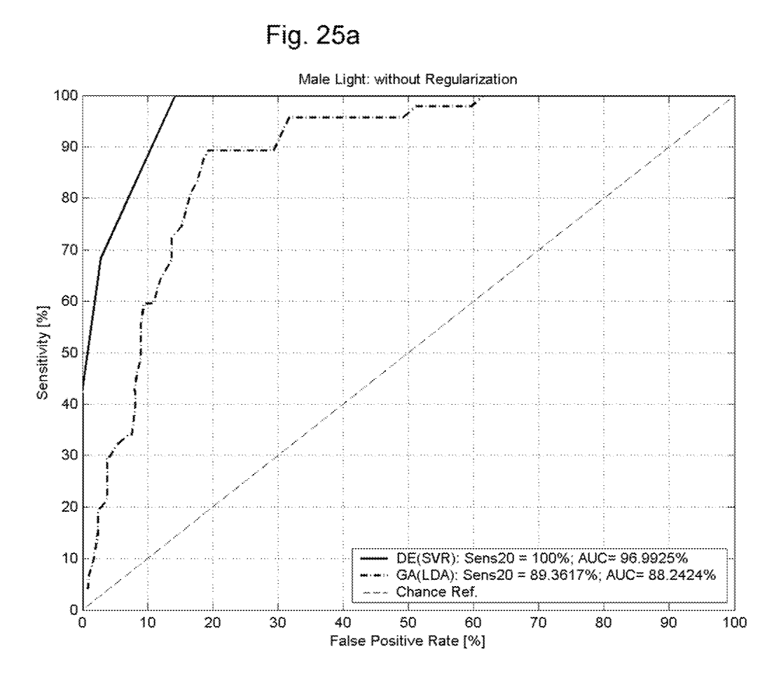

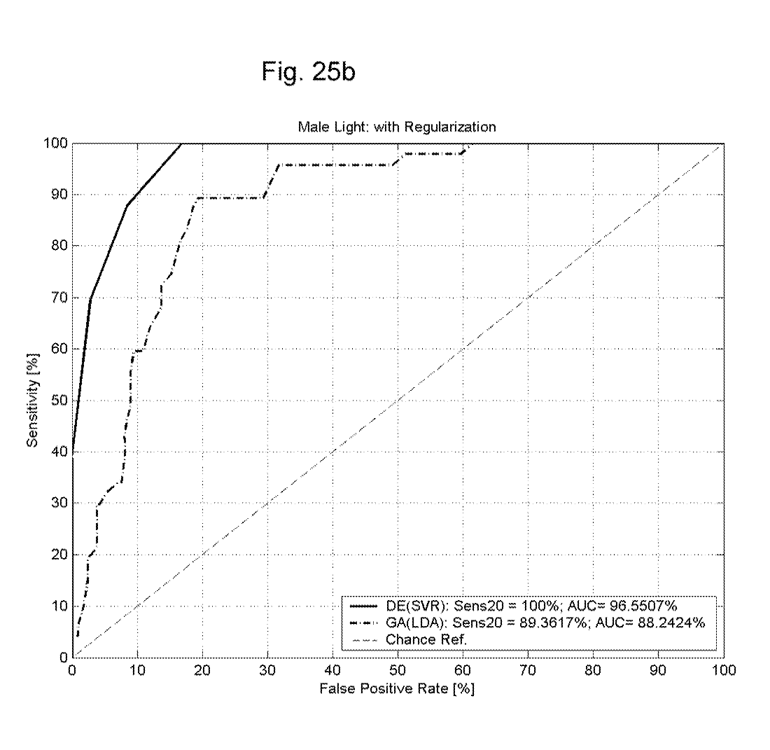

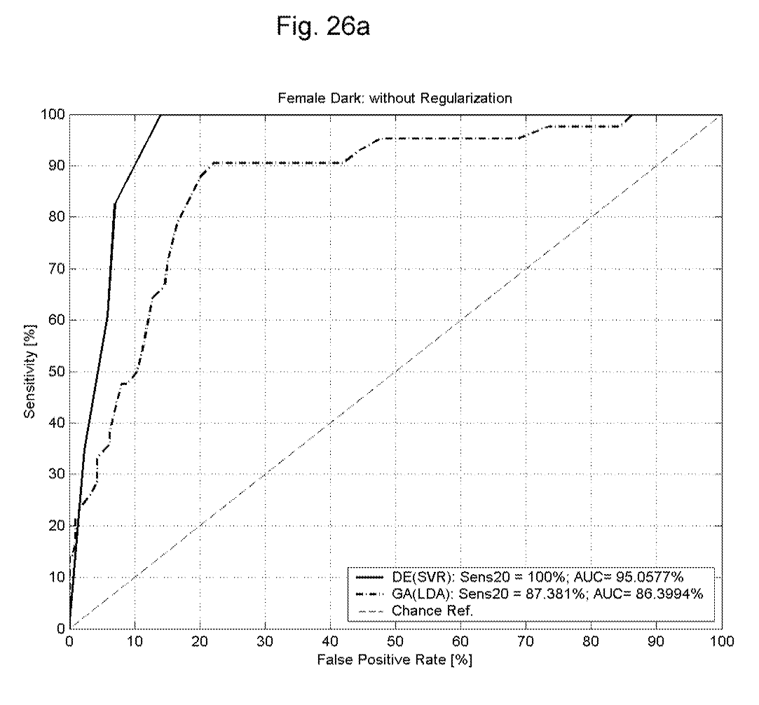

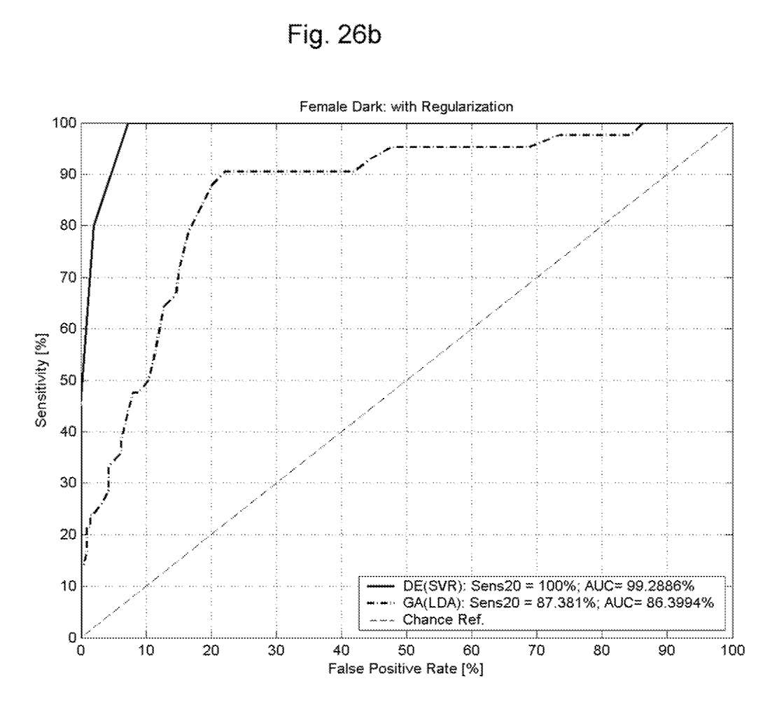

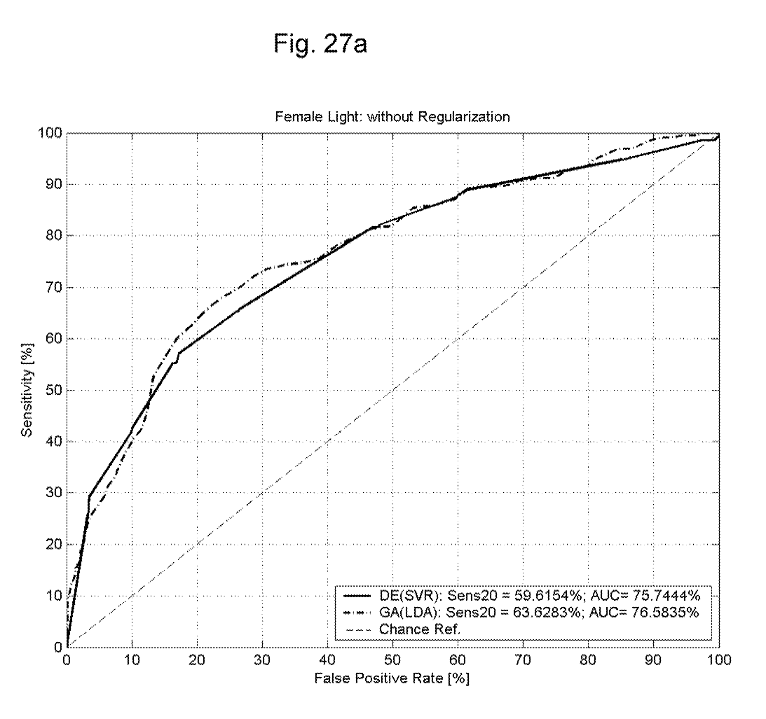

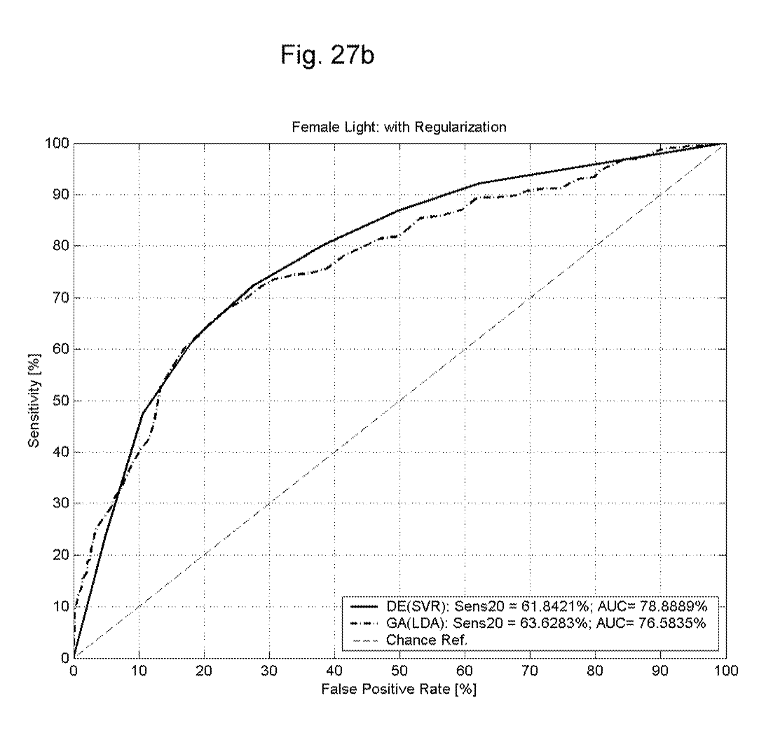

The results shown in FIG. 23-27 illustrate the effect of data regularization of the type described on the skin fluorescence spectra in terms of sensitivity to disease with respect to SVR classification. FIG. 23 illustrates aggregate results. FIG. 24 illustrates results for an individual sub-model for male/dark skin. FIG. 25 illustrates results for an individual sub-model for male/light skin. FIG. 26 illustrates results for an individual sub-model for female/dark skin. FIG. 27 illustrates results for an individual sub-model for female/light skin. Both the LDA and SVR methodologies involved tuning parameters (for the data normalization as well as the classification algorithm itself) and were found via the use of a Genetic Algorithm for the case of LDA and via the use of a technique known as Differential Evolution for the case of SVR. See, e.g., Differential Evolution: A Practical Approach to Global Optimization, Price et al, Springer 2005. These are respectively referred to as GA(LDA) and DE(SVR) wrapper approaches. The DE(SVR) results were generated by combining together the standardized scores of all the SVR sub-models. The results for GA(LDA) were similarly produced from the sub-models. Also shown is the weighted average of the sensitivities for all the sub-models for SVR (weighted by the number of points in each submodel), which is expected to be similar to the DE(SVR) curve and is shown as a reasonable check on the results.

Details of DE(SVR) Based Classification Methodology

The following describes a methodology for producing an empirically stable nonlinear disease classifier for spectral response measurements in general (e.g., fluorescence of the skin, etc.) but can also be used with non-spectral data. Let xᵢ denote one of a set XₘεX of N spectral measurement row vectors such that

Xₘ={x₁,x₂,x₃, . . . ,xᵢ, . . . ,x_{N}}ₘε [symbol] ^{N+D},

where Xₘ denotes a given cross validation fold (subset) of the original data set X and each column (i.e., each of the D response dimensions) is standardized to unit variance and zero mean; and let yᵢ be one of N corresponding binary class labels

yₘ={y₁,y₂,y₃, . . . ,yᵢ, . . . ,y_{N}}ₘε [symbol] ^{N}

for each xᵢ, such that

yᵢ=+1←Disease Positive

yᵢ=−1←Disease Negative

defines the two disease state classes for the data.

For each Xₘ one computes the Singular Value Decomposition such that

$${\{ \begin{matrix}X = {USV}^{T} \\ XV = US\end{matrix} \}}_{m}$$

Then, imposing a filter factor regularization matrix Fₘ, we have

$${\{ \begin{matrix}X ( VF ) = U ( SF ) \\ X \tilde{V} - U \tilde{S}\end{matrix} \}}_{m}$$

with Fₘ defined as

$${F}_{m} = {diag [ \frac{{d}_{j}}{{d}_{j} + γ} ]}_{m}$$

which is a K×K diagonal matrix with K=rank(U); j denotes the j^{th} of the K total left singular (column) vectors {uⱼεU}ₘ [uⱼ is also referred to as an SVD factor];

$${\{ {d}_{j} ≡ \frac{\vert {u}_{j}^{+} - {u}_{j}^{-} \vert}{{s}^{2} ( {u}_{j}^{+} ) + {s}^{2} ( {u}_{j}^{-} )} \}}_{m}$$

is the Fisher distance between the disease-positive labeled points {uⱼ⁺}ₘ and the disease-negative labeled points {uⱼ⁻}ₘ for each SVD factor; and s² denotes the variance.

In this way the SVD factors are weighted relative to each other according to disease separation. Those factors with highest disease separation are treated preferentially. The tuning parameter γ determines the degree to which the SVD factors are treated differently.

At this point a classification procedure known variously as Kernel Ridge Regression (KRR) or Support Vector Regression (SVR) is employed as follows. Letting xᵢ←xᵢ^{m}, the problem is to minimize

$$H = {\sum }_{i = 1}^{N} V ( {y}_{i} - f ( {x}_{i} ) ) + \frac{λ}{2} {‖ f ‖}^{2}$$

with respect to the set of coefficients {fₚ}, given that

$$f ( {x}_{i} ) = {\sum }_{p = 1}^{M} {f}_{p} {h}_{p} ( {x}_{i} )$$

is the Hilbert space expansion of a solution function ƒ in the basis set {hₘ}, and

$${‖ f ‖}^{2} = {\sum }_{p = 1}^{M} {f}_{p}^{2}$$

is the norm of f.
V is an error function, which was chosen to be

$$V ( r ) = \{ \begin{matrix}0 , & if \vert r \vert < ɛ \\ \vert r \vert - ɛ , & otherwise\end{matrix}$$

and λ is another tuning parameter.

Given the form of V above, the solution of equation (1) can be written as

$$\begin{matrix}f ( x ) = {\sum }_{m = 1}^{M} {f}_{p} {h}_{p} ( {x}_{i} ) \\ = {\sum }_{i = 1}^{N} {a}_{i} K ( x , {x}_{i} )\end{matrix}$$

The kernel function K was chosen to be

$$K ( x , {x}_{i} ) = exp [ - \frac{{‖ x - {x}_{i} ‖}^{2}}{2 {σ}^{2}} ]$$

which is known as the radial basis function.

In general, only a number of the coefficients {αᵢ} in the solution f(x) will not be zero. The corresponding data vectors xᵢ are known as support vectors and represent the data points which together are sufficient to represent the entire data set. Depending on the relative fraction of the support vectors that make up the data set, the solution of SVR can be less dependent on outliers and less dependent on the covariance structure of the entire data set. In this sense, the SVR method tries to find the maximum amount of data-characterizing information in the least number of data points. This is in contrast to, for example, Linear Discriminant techniques which are dependent on the covariance of the data set, which involves all the points used in the calibration.

Those skilled in the art will recognize that the present invention can be manifested in a variety of forms other than the example embodiments described and contemplated herein. Accordingly, departures in form and detail can be made without departing from the scope and spirit of the present invention as described in the appended claims.

1. A method for determining the presence of coronary artery calcification in an individual, comprising:
(a) providing a spectroscopic apparatus configured to measure the skin fluorescence of the individual;
(b) detecting the intrinsic skin fluorescence of the individual with the spectroscopic apparatus; and
(c) determining a measure of coronary artery calcification from the intrinsic fluorescence.

2. A method as in claim 1, wherein the measure of coronary artery calcification is a quantitative measurement.

3. A method as in claim 1, wherein the measure of coronary artery calcification is a qualitative measurement.

4. A method as in claim 1, further comprising determining one or more additional factors concerning the individual, and wherein determining a measure of coronary artery calcification comprises determining a measure of coronary artery calcification from the intrinsic fluorescence and from the one or more additional factors.

5. A method as in claim 4, wherein the one or more additional factors comprises one or more of the following: age of the subject, gender, ethnicity, current smoking status, previous smoking status, pack years smoking status, current smoking status times duration of smoking, renal function, estimated glomerular filtration rate, albumin excretion ratio, waist circumference, waist-to-hip ratio, BMI, systolic and/or diastolic blood pressure, use of blood pressure medication, diagnosis of hypertension, lipid levels, use of cholesterol medication, fasting glucose concentration, glycosylated hemoglobin concentration (HbA1c), casual glucose concentration, glucose concentration in response to a glucose challenge test, fructosamine, 1,5-anhydro-D-glucitol, c-reactive .protein, other markers of inflammation or markers of oxidative stress.

6. A method of classifying an individual for risk of coronary artery calcification, coronary artery disease, or a combination thereof, comprising determining a measure of the skin intrinsic fluorescence of the individual, and classifying the individual risk from a model relating skin intrinsic fluorescence and risk.

7. A method as in claim 6, further comprising determining one or more additional factors concerning the individual, and wherein classifying the individual risk from a model relating skin intrinsic fluorescence and risk comprises classifying the individual risk from a model relating (a) skin intrinsic fluorescence and the one or more other factors and (b) risk.

8. A method as in claim 7, wherein the one or more additional factors comprises one or more of the following: age of the subject, gender, ethnicity, smoking status, previous smoking status, pack years smoking status, current smoking status times duration of smoking, renal function, estimated glomerular filtration rate, albumin excretion ratio, waist circumference, waist-to-hip ratio, BMI, systolic and/or diastolic blood pressure, use of blood pressure medication, diagnosis of hypertension, lipid levels, use of cholesterol medication, fasting glucose concentration, glycosylated hemoglobin concentration (HbA1c), casual glucose concentration, glucose concentration in response to a glucose challenge test, fructosamine, 1,5-anhydro-D-glucitol, c-reactive protein, other markers of inflammation or markers of oxidative stress.

9. A method as in claim 1, wherein the spectroscopic apparatus comprises:
(a) an illumination system adapted to produce light at a plurality of broadband wavelength ranges;
(b) an optical probe adapted to receive broadband light from the illumination system and transmit the broadband light to in vivo tissue, and to receive light diffusely reflected in response to the broadband light, emitted from the in vivo tissue by fluorescence thereof in response to the broadband light, or a combination thereof;
(c) a calibration device which can be periodically placed in optical communication with the optical probe;
(d) a spectrograph adapted to receive the light from the optical probe and produce a signal representative of spectral properties of the light; and
(e) an analysis system adapted to determine a property of the in vivo tissue from the spectral properties signal.

10. The method of claim 9, wherein the calibration device comprises a fluorescent material.

11. The method of claim 10, wherein the calibration device substantially blocks ambient light from reaching the optical probe when the calibration device is placed in optical communication with the optical probe.

12. The method of claim 10, wherein the calibration device comprises a reflective material.

13. The method of claim 10, wherein the calibration device comprises a housing that defines a chamber having walls, a fluorescent material disposed on at least a portion of the walls, a reflective material disposed on at least a portion of the walls, wherein the housing has a first end adapted to substantially prevent ambient light from reaching the optical probe when the chamber if placed in optical communication with the optical probe.

14. The method of claim 13, wherein the chamber has a substantially spherical shape.

15. The method of claim 13, wherein the chamber has a cross-section that provides uniform illumination of the optical probe with light reflected by the calibration device as well as fluorescence emitted by the calibration device.

16. The method of claim 9, wherein the spectroscopic apparatus further comprises an operator display adapted to communicate information concerning the determined tissue property, where the display mounts with the apparatus such that the display can be adjusted in two angular dimensions, and wherein the operator display comprises a touchscreen adapted to accept input from an operator responsive to touch of the screen by the operator.

17. The method of claim 16, wherein the display can be adjusted such that a human whose tissue is being sampled by the apparatus cannot see the display.

18. The method of claim 9, wherein the spectroscopic apparatus further comprises an arm positioning element adapted to position a human arm relative to the optical probe such that the optical probe communicates light with a portion of the forearm, and wherein the arm positioning element has a concave shape that interfaces the forearm with the optical probe in a manner that substantially prevents ambient light from being detected by the optical probe.

19. (canceled)

20. (canceled)

21. The method of claim 9, wherein the spectrograph is adapted to produce a spectrum that is substantially free of ghost images and stray light.

22. The method of claim 21, wherein the spectrograph comprises a back-illuminated CCD image sensor.

23. The method of claim 22, wherein the back-illuminated CCD is oriented non-perpendicular to the axis of incident light thereon.

24. The method of claim 9, wherein the spectrograph comprises an out-of-plane Littrow spectrograph.

25. The method of claim 21, wherein spectrograph comprises a front-illuminated CCD detection element.

26. (canceled)

27. A method of determining the CAC value of a subject, or detecting whether a subject has a clinically significant CAC above a predetermined threshold, comprising:
determining the intrinsic fluorescence of a portion of the skin of the subject;
determining the CAC value of a subject, or detecting whether a subject has a clinically significant CAC above a predetermined threshold, from the intrinsic fluorescence.

28. A method as in claim 27, wherein determining the intrinsic fluorescence comprises determining the sum of intrinsic fluorescence of the skin at each of a plurality of wavelengths.

29. A method as in claim 28, wherein the step of determining the CAC value, or detecting a clinically significant CAC, comprises using a multivariate model that relates CAC to the sum of intrinsic fluorescence and one or more of the following: age of the subject, gender, ethnicity, diagnosed type 1 or type 2 diabetes, skin tone, skin tone quantified by the sum of the skin reflectance across the fluorescence emission band, current smoking status, previous smoking status, pack years smoking status, current smoke smoking status times duration of smoking, renal function, estimated glomerular filtration rate, albumin excretion ratio, waist circumference, waist-to-hip ratio, BMI, systolic and/or diastolic blood pressure, use of blood pressure medication, diagnosis of hypertension, lipid levels, use of cholesterol medication, fasting glucose concentration, glycosylated hemoglobin concentration (HbA1c), casual glucose concentration, glucose concentration in response to a glucose challenge test, fructosamine, 1,5-anhydro-D-glucitol, c-reactive protein, other markers of inflammation or markers of oxidative stress.

30. (canceled)

31. A method as in claim 29, wherein the multivariate model accepts as input an information vector comprising intrinsic fluorescence and one or more of the following: age of the subject, gender, ethnicity, diagnosed type 1 or type 2 diabetes, skin tone as quantified by the sum of the skin reflectance across the fluorescence emission band, current smoking status, previous smoking status, pack years smoking status, current smoking status times duration of smoking, renal function, estimated glomerular filtration rate, albumin excretion ratio, waist circumference, waist-to-hip ratio, BMI, systolic and/or diastolic blood pressure, use of blood pressure medication, diagnosis of hypertension, lipid levels, use of cholesterol medication, fasting glucose concentration, glycosylated hemoglobin concentration (HbA1c), casual glucose concentration, glucose concentration in response to a glucose challenge test, fructosamine, 1,5-anhydro-D-glucitol, c-reactive protein, other markers of inflammation or markers of oxidative stress.

32. (canceled)

33. A method as in claim 27, wherein using a multivariate model comprises selecting a multivariate model from a plurality of multivariate models based on one or more of the following: the subject's gender, the subject's age, the subject's ethnicity, or the subject's skintone.

34. A method as in claim 27, wherein using a multivariate model comprises combining the outputs of a plurality of multivariate models.

35. (canceled)

36. (canceled)
